SEQUENCE LISTING (60 sequences)

SEQ ID NO: 1 - DNA, 42 bp, Artificial Sequence
  Synthetic Oligonucleotide
acttcgtcagtaacggacgtcctaggagaagagctgcatcca

SEQ ID NO: 2 - DNA, 42 bp, Artificial Sequence
  Synthetic Oligonucleotide
gagtcgaggtcatatcgtgtcctaggagaagagctgcatcca

SEQ ID NO: 3 - DNA, 39 bp, Artificial Sequence
  Synthetic Oligonucleotide
acttcgtcagtaacggacgctcctacccgcccatcaccc

SEQ ID NO: 4 - DNA, 39 bp, Artificial Sequence
  Synthetic Oligonucleotide
gagtcgaggtcatatcgtgctcctacccgcccatcaccc

SEQ ID NO: 5 - DNA, 41 bp, Artificial Sequence
  Synthetic Oligonucleotide
acttcgtcagtaacggacggagagaacttggtgcctcttcc

SEQ ID NO: 6 - DNA, 41 bp, Artificial Sequence
  Synthetic Oligonucleotide
gagtcgaggtcatatcgtggagagaacttggtgcctcttcc

SEQ ID NO: 7 - DNA, 37 bp, Artificial Sequence
  Synthetic Oligonucleotide
acttcgtcagtaacggacgcgaacccagacccccagg

SEQ ID NO: 8 - DNA, 37 bp, Artificial Sequence
  Synthetic Oligonucleotide
gagtcgaggtcatatcgtgcgaacccagacccccagg

SEQ ID NO: 9 - DNA, 41 bp, Artificial Sequence
  Synthetic Oligonucleotide
acttcgtcagtaacggacgccagcaaagaatggctcaagaa

SEQ ID NO: 10 - DNA, 41 bp, Artificial Sequence
  Synthetic Oligonucleotide
gagtcgaggtcatatcgtgccagcaaagaatggctcaagaa

SEQ ID NO: 11 - DNA, 42 bp, Artificial Sequence
  Synthetic Oligonucleotide
acttcgtcagtaacggacgtcacctttctccaaaggcagatg

SEQ ID NO: 12 - DNA, 42 bp, Artificial Sequence
  Synthetic Oligonucleotide
gagtcgaggtcatatcgtgtcacctttctccaaaggcagatg

SEQ ID NO: 13 - DNA, 39 bp, Artificial Sequence
  Synthetic Oligonucleotide
acttcgtcagtaacggacaatccttccttgcttccagcg

SEQ ID NO: 14 - DNA, 39 bp, Artificial Sequence
  Synthetic Oligonucleotide
gagtcgaggtcatatcgtaatccttccttgcttccagcg

SEQ ID NO: 15 - DNA, 38 bp, Artificial Sequence
  Synthetic Oligonucleotide
acttcgtcagtaacggacagcgcggcactcaggtacct

SEQ ID NO: 16 - DNA, 38 bp, Artificial Sequence; identical to SEQ ID NO: 15
  Synthetic Oligonucleotide

SEQ ID NO: 17 - DNA, 40 bp, Artificial Sequence
  Synthetic Oligonucleotide
acttcgtcagtaacggacatgttcctgtgctccatgatgc

SEQ ID NO: 18 - DNA, 40 bp, Artificial Sequence
  Synthetic Oligonucleotide
gagtcgaggtcatatcgtatgttcctgtgctccatgatgc

SEQ ID NO: 19 - DNA, 63 bp, Artificial Sequence
  Synthetic Oligonucleotide
gtcgctgatcttacaacactattacatgcctattgacgtgaggcggtctgcctatagtgagtc

SEQ ID NO: 20 - DNA, 41 bp, Artificial Sequence
  Synthetic Oligonucleotide
acttcgtcagtaacggacgtccctggagtaaaactgcggtc

SEQ ID NO: 21 - DNA, 41 bp, Artificial Sequence
  Synthetic Oligonucleotide
gagtcgaggtcatatcgtgtccctggagtaaaactgcggtc

SEQ ID NO: 22 - DNA, 63 bp, Artificial Sequence
  Synthetic Oligonucleotide
aaaatgtccctccgttcttatctagatcgcaaaagtgtctcggaagtctgcctatagtgagtc

SEQ ID NO: 23 - DNA, 39 bp, Artificial Sequence
  Synthetic Oligonucleotide
acttcgtcagtaacggacgggtttctccaaggcccttca

SEQ ID NO: 24 - DNA, 39 bp, Artificial Sequence
  Synthetic Oligonucleotide
gagtcgaggtcatatcgtgggtttctccaaggcccttca

SEQ ID NO: 25 - DNA, 62 bp, Artificial Sequence
  Synthetic Oligonucleotide
gaagacgatgacctcgacttcatacgcgaattgatagaagctcggtctgcctatagtgagtc

SEQ ID NO: 26 - DNA, 41 bp, Artificial Sequence
  Synthetic Oligonucleotide
acttcgtcagtaacggactgcaactctgctcaggatcattt

SEQ ID NO: 27 - DNA, 41 bp, Artificial Sequence
  Synthetic Oligonucleotide
gagtcgaggtcatatcgttgcaactctgctcaggatcattt

SEQ ID NO: 28 - DNA, 62 bp, Artificial Sequence
  Synthetic Oligonucleotide
ccagaacaaatgtatgaggaattcactcaaggccgttagctgtggtctgcctatagtgagtc

SEQ ID NO: 29 - DNA, 43 bp, Artificial Sequence
  Synthetic Oligonucleotide
acttcgtcagtaacggacggaagcttcgtcatcttggactcag

SEQ ID NO: 30 - DNA, 43 bp, Artificial Sequence
  Synthetic Oligonucleotide
gagtcgaggtcatatcgtggaagcttcgtcatcttggactcag

SEQ ID NO: 31 - DNA, 66 bp, Artificial Sequence
  Synthetic Oligonucleotide
aaaagtgattctagcaatagtgattttactgcgctcctaattggcaccgtctgcctatagtgagtc

SEQ ID NO: 32 - DNA, 38 bp, Artificial Sequence
  Synthetic Oligonucleotide
acttcgtcagtaacggacccaaatgcgctggacacatt

SEQ ID NO: 33 - DNA, 38 bp, Artificial Sequence
  Synthetic Oligonucleotide
gagtcgaggtcatatcgtccaaatgcgctggacacatt

SEQ ID NO: 34 - DNA, 62 bp, Artificial Sequence
  Synthetic Oligonucleotide
gtacctttctgatgatgaggacggaacagcttacgactttgcgggtctgcctatagtgagtc

SEQ ID NO: 35 - DNA, 47 bp, Artificial Sequence
  Synthetic Oligonucleotide
tcctaggagaagagctgcatccatggacaacaacagtaaatttgcta

SEQ ID NO: 36 - DNA, 43 bp, Artificial Sequence
  Synthetic Oligonucleotide
ctcctacccgcccatcacccgctcacagtacgactacaccgac

SEQ ID NO: 37 - DNA, 47 bp, Artificial Sequence
  Synthetic Oligonucleotide
ggagagaacttggtgcctcttccactctggagtgaagttaatgaaag

SEQ ID NO: 38 - DNA, 42 bp, Artificial Sequence
  Synthetic Oligonucleotide
cgaacccagacccccagggctgagttaaaggagaaattcacc

SEQ ID NO: 39 - DNA, 46 bp, Artificial Sequence
  Synthetic Oligonucleotide
ccagcaaagaatggctcaagaagtactgacacatttaaaggagcat

SEQ ID NO: 40 - DNA, 47 bp, Artificial Sequence
  Synthetic Oligonucleotide
tcacctttctccaaaggcagatgtgaagaacttgatgtcttatgtgg

SEQ ID NO: 41 - DNA, 45 bp, Artificial Sequence
  Synthetic Oligonucleotide
aatccttccttgcttccagcgttactacagcagataggtcgagag

SEQ ID NO: 42 - DNA, 43 bp, Artificial Sequence
  Synthetic Oligonucleotide
agcgcggcactcaggtacctgacaatgatgagcagtttgtacc

SEQ ID NO: 43 - DNA, 46 bp, Artificial Sequence
  Synthetic Oligonucleotide
atgttcctgtgctccatgatgcagtcgctgatcttacaacactatt

SEQ ID NO: 44 - DNA, 44 bp, Artificial Sequence
  Synthetic Oligonucleotide
tccctggagtaaaactgcggtcaaaaatgtccctccgttcttat

SEQ ID NO: 45 - DNA, 41 bp, Artificial Sequence
  Synthetic Oligonucleotide
ggtttctccaaggcccttcaggaagacgatgacctcgactt

SEQ ID NO: 46 - DNA, 46 bp, Artificial Sequence
  Synthetic Oligonucleotide
tgcaactctgctcaggatcatttaccagaacaaatgtatgaggaat

SEQ ID NO: 47 - DNA, 50 bp, Artificial Sequence
  Synthetic Oligonucleotide
gaagcttcgtcatcttggactcagtaaaagtgattctagcaatagtgatt

SEQ ID NO: 48 - DNA, 43 bp, Artificial Sequence
  Synthetic Oligonucleotide
ccaaatgcgctggacacattcgtacctttctgatgatgaggac

SEQ ID NO: 49 - DNA, 23 bp, Homo sapiens
agcagcattgtacagggctatga

SEQ ID NO: 50 - DNA, 21 bp, Homo sapiens
tccctgtcctccaggagctca

SEQ ID NO: 51 - DNA, 23 bp, Homo sapiens
tatggcactggtagaattcactg

SEQ ID NO: 52 - DNA, 22 bp, Homo sapiens
tttggcaatggtagaactcaca

SEQ ID NO: 53 - DNA, 23 bp, Homo sapiens
agctacattgtctgctgggtttc

SEQ ID NO: 54 - DNA, 23 bp, Homo sapiens
actccatttgttttgatgatgga

SEQ ID NO: 55 - DNA, 59 bp, Artificial Sequence
  Synthetic Oligonucleotide
acttcgtcagtaacggactccagtagcgactagcccgtcagcagcattgtacagggcta

SEQ ID NO: 56 - DNA, 58 bp, Artificial Sequence
  Synthetic Oligonucleotide
acttcgtcagtaacggactataccggcctaagcactcgcaccctgtcctccaggagct

SEQ ID NO: 57 - DNA, 59 bp, Artificial Sequence
  Synthetic Oligonucleotide
acttcgtcagtaacggacaatgttgacccggatctcgtccatggcactggtagaattca

SEQ ID NO: 58 - DNA, 59 bp, Artificial Sequence
  Synthetic Oligonucleotide
acttcgtcagtaacggacactagccctcgcatagcttgcgtttggcaatggtagaactc

SEQ ID NO: 59 - DNA, 58 bp, Artificial Sequence
  Synthetic Oligonucleotide
acttcgtcagtaacggacgcgcaattccctcgatcttacgctacattgtctgctgggt

SEQ ID NO: 60 - DNA, 60 bp, Artificial Sequence
  Synthetic Oligonucleotide
acttcgtcagtaacggacgtaggtcccggacgtaatcaccactccatttgttttgatgat

(19) United States
(12) Patent Application Publication
(10) Pub. No.: US 2011/0230361 A1
(43) Pub. Date: Sep. 22, 2011

(54) PROSTATE CANCER BIOMARKERS TO PREDICT RECURRENCE AND METASTATIC POTENTIAL

(75) Inventors: Carlos Moreno, Atlanta, GA (US); Adeboye Osunkoya, Lilburn, GA (US); Wei Zhou, Lilburn, GA (US); Brian Leyland-Jones, Atlanta, GA (US); Qi Long, Atlanta, GA (US); Brent A Johnson, Atlanta, GA (US)

(73) Assignee: EMORY UNIVERSITY, Atlanta, GA (US)

(21) Appl. No.: 13/129,122

(22) PCT Filed: Nov. 13, 2009
(86) PCT No.: PCT/US09/64384

§ 371 Date: Jun. 13, 2011

Related U.S. Application Data
(60) Provisional application No. 61/114,658, filed on Nov. 14, 2008.

(51) Int. Cl. C40B 30/00; C12Q 1/68; C40B 40/06

(52) U.S. Cl. 506/7; 435/6.14; 435/6.12; 506/16

(57) ABSTRACT

Described herein are methods for predicting the recurrence, progression, and metastatic potential of a prostate cancer in a subject. For example, the method comprises detecting in a sample from a subject one or more biomarkers selected from the group consisting of FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION. The method can further comprise detecting in a sample from a subject one or more biomarkers selected from the group consisting of miR-103, miR-339, miR-183, miR-182, miR-136, and miR-221. An increase or decrease in one or more biomarkers as compared to a standard indicates a recurrent, progressive, or metastatic prostate cancer.

### CROSS-REFERENCE TO RELATED APPLICATION

This application claims the benefit of U.S. Provisional Application No. 61/114,658, filed on Nov. 14, 2008.

### STATEMENT REGARDING FEDERALLY FUNDED RESEARCH

The invention was made with government support under Grant Nos. RO1CA106826 and K22CA96560 from the National Institutes of Health. The government has certain rights in this invention.

### BACKGROUND

Prostate cancer is the most commonly diagnosed noncutaneous neoplasm and second most common cause of cancer-related mortality in Western men. One of the important challenges in current prostate cancer research is to develop effective methods to determine whether a patient is likely to progress to the aggressive, metastatic disease in order to aid clinicians in deciding the appropriate course of treatment. The current standard for pathological evaluation of the status of prostate cancer patients is the Gleason score. The Gleason score is calculated based on summing the grades of glandular architecture of the two most prevalent histological components of the tumor. However, it is currently very difficult to predict the outcome of patients based solely on the Gleason score.

In medical studies, it is of substantial interest to conduct feature selection using high dimensional biomarker data such as gene expression data, when the outcome of interest may be censored, e.g., censored time to the development or recurrence of a disease. Subsequently, these selected features can be used to predict the risk of developing a disease. In the presence of clinical variables that have been established as the risk factors of a disease, it is preferred to use a feature selection procedure that also adjusts for these clinical variables.

### SUMMARY

Provided are methods of predicting the recurrence, progression, and/or metastatic potential of a prostate cancer in a subject. Specifically, the methods comprise selecting a subject at risk of recurrence, progression or metastasis of prostate cancer, and detecting in a sample from the subject one or more biomarkers selected from the group consisting of FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION to create a biomarker profile. An increase or decrease in one or more of the biomarkers as compared to a standard indicates a prostate cancer that is prone to recur, progress, and/or metastasize. The sample can, for example, comprise prostate tumor tissue. The method further comprises detecting one or more biomarkers selected from the group consisting of miR-103, miR-339, miR-183, miR-182, miR-136, and miR-221.

Also provided are methods of predicting the recurrence, progression, and/or metastatic potential of a prostate cancer in a subject, the methods comprising selecting a subject at risk of recurrence, progression, or metastasis of prostate cancer, and detecting in a sample from a subject one or more biomarkers selected from the group consisting of miR-103, miR-339, miR-183, miR-182, miR-136, and miR-221 to create a biomarker profile. An increase or decrease in one or more of the biomarkers as compared to a standard indicates a prostate cancer that is prone to recur, progress, and/or metastasize.

Also provided are methods of treating a subject with prostate cancer comprising modifying the treatment regimen of the subject based on the results of the method of predicting the recurrence, progression, and/or metastatic potential of a prostate cancer in a subject. The treatment regiment is modified to be aggressive based on an increase in one or more biomarkers selected from the group consisting of CLNS1A, XPO1, LETMD1, RAD23B, TMPRSS2 ETV1 FUSION, ABCC3, APC, CHES1, FRZB, HSPG2, miR-103, miR-339, miR-183, and miR-182 as compared to a standard, and a decrease in one or more biomarkers selected from a group consisting of FOXO1A, SOX9, PTGDS, EDNRA, miR-136, and miR-221 as compared to a standard.

Also provided are kits comprising primers to detect the expression of biomarkers selected from the group consisting of FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION, and primers to detect the expression of biomarkers selected from the group consisting of miR-103, miR-339, miR-183, miR-182, miR-136, and miR-221.

Also provided are methods of predicting recurrence potential of a disease. The methods comprise detecting gene expression profiles in subjects with the disease; detecting sets of clinical variables associated with the disease in subjects with the disease; parametrically modeling the gene expression profile and non-parametrically modeling the set of clinical variables; and selecting gene expression profiles and clinical variables consistent with a selected recurrence potential, wherein the selection step comprises Lasso type estimation.

Further provided are computer systems for predicting recurrence potential of a disease. The computer systems comprise (a) a memory on which is stored a database comprising (i) a plurality of gene expression profiles for the disease, wherein each gene expression profile comprises a plurality of values, each value representing the expression level of a gene; (ii) a plurality of sets of clinical variables associated with the disease; and (iii) a descriptor associated with recurrence potential of the disease, wherein the descriptor is based on a combination of the gene expression profiles and the sets of clinical variables; and (b) a processor having computer-executable code for effecting the following steps (i) parametrically modeling the gene expression profiles and non-parametrically modeling the sets of clinical variables; (ii) selecting gene expression profiles and sets of clinical variables consisting with a reference recurrence potential; and (iii) outputting the descriptor stating the recurrence potential for the disease based on the combination of the gene expression profile and the set of clinical variables.

### DESCRIPTION OF DRAWINGS

FIG. 1 shows a Kaplan-Meier plot for the prediction of the recurrence, progression, and/or metastatic potential of prostate cancer based on the differential expression of the FOXO1A, SOX9, CLNS1A, PTGDS, XPO1, LETMD1, RAD23B, and TMPRSS2_ETV1 FUSION protein coding genes in samples collected from 71 patients. (p=0.001).

FIG. 2 shows a Kaplan-Meier plot for the prediction of the recurrence, progression, and/or metastatic potential of prostate cancer based on the differential expression of the FOXO1A, SOX9, CLNS1A, PTGDS, XPO1, LETMD1, RAD23B, and TMPRSS2_ETV1 FUSION protein coding genes in samples collected from 46 patients with a Gleason score of 7. (p=0.022).

FIG. 3 shows a Kaplan-Meier plot for the prediction of the recurrence, progression, and/or metastatic potential of prostate cancer based on the differential expression of the miR-103, miR-339, miR-183, miR-182, miR-136, and miR-221 genes in samples collected from 71 patients. (p=0.001).

FIG. 4 shows a Kaplan-Meier plot for the prediction of the recurrence, progression, and/or metastatic potential of prostate cancer based on the differential expression of the miR-103, miR-339, miR-183, miR-182, miR-136, and miR-221 genes in samples collected from 46 patients with a Gleason score of 7. (p=0.032).

FIG. 5 shows a Kaplan-Meier plot for the prediction of recurrence, progression, and/or metastatic potential of prostate cancer based on the differential expression of ABCC3, APC, CHES1, EDNRA, FRZB, and HSPG2 protein coding genes in samples collected from 71 patients. (p=6.24×10⁻⁵)

FIG. 6 shows a graph demonstrating the estimated effect of PSA level on prediction of the recurrence, progression, and/or metastatic potential of prostate cancer.

FIG. 7 shows a graph demonstrating the cumulative distribution function (CDF) of the p-values for evaluating the prediction performance. A: the Lasso-PL using all data; B: the Lasso-L using all data; C: the usual AFT model using all data; D: the partly linear AFT model using only the clinical variables; E: the linear AFT model using only the clinical variables.

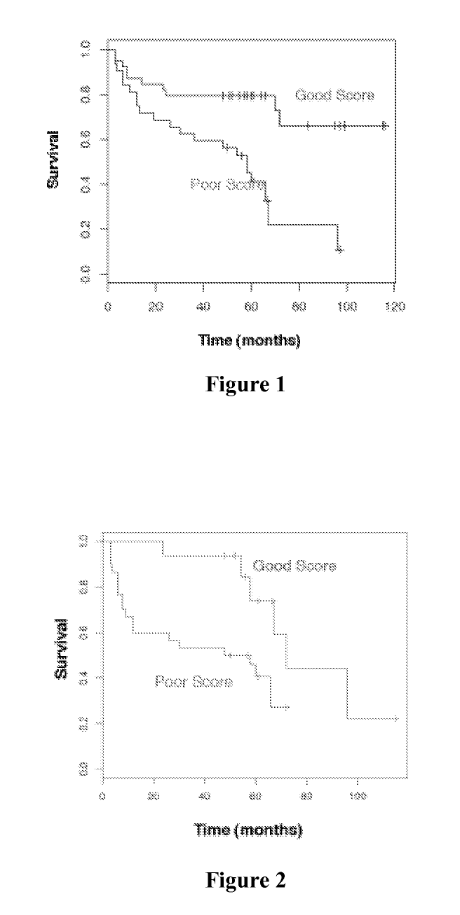

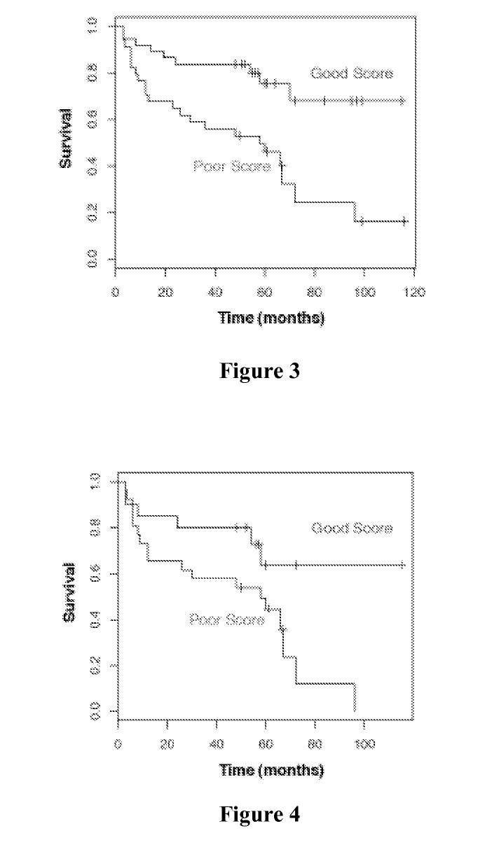

### DETAILED DESCRIPTION

Described herein are methods for predicting the recurrence, progression, and/or metastatic potential of a prostate cancer in a subject. The methods comprise selecting a subject at risk of recurrence, progression, or metastasis of prostate cancer, and detecting in a sample from a subject one or more biomarkers selected from the group consisting of FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION to create a biomarker profile. An increase or decrease in one or more of the biomarkers as compared to a standard indicates a prostate cancer that is prone to recur, progress, and/or metastasize. Optionally, the sample comprises prostate tumor tissue.

Optionally, multiple biomarkers are detected. Detection can comprise identifying an RNA expression pattern. An increase in one or more of the biomarkers selected from the group consisting of CLNS1A, XPO1, LETMD1, RAD23B, TMPRSS2_ETV1 FUSION, ABCC3, SPC, CHES1, FRZB, and HSPG2 as compared to a standard indicates a prostate cancer that is prone to recur, progress, and/or metastasize. A decrease in one or more of the biomarkers selected from the group consisting of FOXO1A, SOX9, EDNRA, and PTGDS as compared to a standard indicates a prostate cancer that is prone to recur, progress, and/or metastasize. Optionally, the detected biomarkers comprise two or more, three or more, four or more, five or more, six or more, seven or more, eight or more, nine or more, ten or more, eleven or more, twelve or more, thirteen or more, or all fourteen biomarkers selected from the group consisting of FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION. For example, the detected biomarkers can comprise FOXO1A and SOX9. Alternatively, the detected biomarkers can comprise TGDS and ABCC3. For example, the detected biomarkers can comprise SOX9, CLNS1A, and TMPRSS2_ETV1 FUSION. Alternatively, the detected biomarkers can comprise PTGDS, XPO1, and CHES1. For example, the detected biomarkers can comprise FOXO1A, PTGDS, XPO1, and RAD23B. Alternatively, the detected biomarkers can comprise CLNS1A, LETMD1, RAD23B, and EDNRA. For example, the selected biomarkers can comprise FOXO1A, SOX9, CLNS1A, PTGDS, and LETMD1. Alternatively, the selected biomarkers can comprise FOXO1A, CLNS1A, PTGDS, XPO1, and FRZB. For example, the selected biomarkers can comprise FOXO1A, CLNS1A, PTGDS, XPO1, LETMD1, and RAD23B. For example, the selected biomarkers can comprise SOX9, CLNS1A, PTGDS, XPO1, LETMD1, RAD23B, and TMPRSS2_ETV1 FUSION. For example, the selected biomarkers can comprise FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION. For example, the selected biomarkers can comprise FOXO1A, SOX9, CLNS1A, XPO1, RAD23B, ABCC3, EDNRA, FRZB, and TMPRSS2_ETV1 FUSION. For example, the selected biomarkers can comprise FOXO1A, SOX9, CLNS1A, PTGDS, XPO1, LETMD1, RAD23B, ABCC3, and APC. For example, the selected biomarkers can comprise FOXO1A, SOX9, CLNS1A, PTGDS, XPO1, LETMD1, RAD23B, ABCC3, APC, and CHES1. For example, the selected biomarkers can comprise FOXO1A, SOX9, CLNS1A, PTGDS, XPO1, LETMD1, RAD23B, ABCC3, APC, EDNRA, HSPG2, and TMPRSS2_ETV1 FUSION. For example, the selected biomarkers can comprise FOXO1A, SOX9, CLNS1A, PTGDS, XPO1, LETMD1, RAD23B, ABCC3, APC, CHES1, EDRNA, FRZB, and HSPG2. Optionally, the selected biomarkers comprise biomarkers selected from the group consisting of FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION.

Optionally, the method further comprises detecting one or more, two or more, three or more, four or more, five or more, or all six biomarkers selected from the group consisting of miR-103, miR-339, miR-183, miR-182, miR-136, and miR-221. For example, the selected biomarker can comprise miR-339. Alternatively, the selected biomarker can comprise miR-182. For example, the selected biomarkers can comprise miR-103 and miR-339. Alternatively, the selected biomarkers can comprise miR-136 and miR-221. For example, the selected biomarkers can comprise miR-103, miR-183, and miR-182. Alternatively, the selected biomarkers can comprise miR-339, miR-136, and miR-221. For example, the selected biomarker can comprise miR-103, miR-339, miR-136, and miR-221. Alternatively, the selected biomarkers can comprise miR-103, miR-183, miR-182, and miR-221. For example, the selected biomarkers can comprise miR-103, miR-339, miR-183, miR-182, and miR-136. Optionally, the method further comprises detecting biomarkers selected from the group consisting of miR-103, miR-339, miR-183, miR-182, miR-136, and miR-221.

Also provided are methods of predicting the recurrence, progression, and/or metastatic potential of a prostate cancer in a subject. The methods comprise selecting a subject at risk of recurrence, progression, or metastasis of prostate cancer, and detecting in a sample from a subject one or more biomarkers selected from the group consisting of miR-103, miR-339, miR-183, miR-182, miR-136, and miR-221 to create a biomarker profile. An increase or decrease in one or more biomarkers as compared to a standard indicates a prostate cancer that is prone to recur, progress, and/or metastasize. Optionally, the sample can comprise prostate tumor tissue.

Optionally, multiple biomarkers are detected. Detection can comprise identifying an RNA expression pattern. An increase in one or more biomarkers selected from the group consisting of miR-103, miR-339, miR-183, and miR-182 as compared to a standard indicates a prostate cancer that is prone to recur, progress, and/or metastasize. A decrease in one or more of the biomarkers selected from miR-136 and miR-221 as compared to a control indicates a prostate cancer that is prone to recur, progress, and/or metastasize. Optionally, the detected biomarkers comprise two or more, three or more, four or more, five or more, or all six biomarkers selected from the group consisting of miR-103, miR-339, miR-183, miR-182, miR-136, and miR-221. For example, the detected biomarkers can comprise miR-136 and miR-221. Alternatively, the detected biomarkers can comprise miR-103 and miR-182. For example, the detected biomarkers can comprise miR-103, miR-339, and miR-183. Alternatively, the detected biomarkers can comprise miR-339, miR-136, and miR-221. For example, the detected biomarkers can comprise miR-103, miR-339, miR-183, and miR-182. Alternatively, the detected biomarkers can comprise miR-183, miR-182, miR-136, and miR-221. For example, the detected biomarkers can comprise miR-339, miR-183, miR-182, miR-136, and miR-221. Optionally, the detected biomarkers comprise biomarkers selected from the group consisting of miR-103, miR-339, miR-183, miR-182, miR-136, and miR-221.

Optionally, the detecting step comprises detecting mRNA levels of the biomarker. The mRNA detection can, for example, comprise reverse-transcription polymerase chain reaction (RT-PCR), quantitative real-time PCR (qRT-PCR), Northern analysis, microarray analysis, and cDNA-mediated annealing, selection, extension, and ligation (DASL®) assay (Illumina, Inc.; San Diego, Calif.). Preferably, the RNA detection comprises the cDNA-mediated annealing, selection, extension, and ligation (DASL®) assay (Illumina, Inc.). Optionally, the detecting step comprises detecting miRNA levels of the biomarker. The miRNA detection can, for example, comprise miRNA chip analysis, Northern analysis, RNase protection assay, in situ hybridization, miRNA expression profiling panels designed for the DASL® assay (Illumina, Inc.), or a modified reverse transcription quantitative real-time polymerase chain reaction assay (qRT-PCR). Preferably the miRNA detection comprises the miRNA expression profiling panels designed for the DASL® assay (Illumina, Inc.). Optionally, the detecting step comprises detecting mRNA and miRNA levels of the biomarker. The analytical techniques used to determine mRNA and miRNA expression are known. See, e.g., Sambrook et al., Molecular Cloning: A Laboratory Manual, 3^{rd} Ed., Cold Spring Harbor Press, Cold Spring Harbor, N.Y. (2001), Yin et al., Trends Biotechnol. 26:70-6 (2008); Wang and Cheng, Methods Mol. Biol. 414:183-90 (2008); Einat, Methods Mol. Biol. 342:139-57 (2006).

Comparing the mRNA or miRNA biomarker content with a biomarker standard includes comparing mRNA or miRNA content from the subject with the mRNA or miRNA content of a biomarker standard. Such comparisons can be comparisons of the presence, absence, relative abundance, or combination thereof of specific mRNA or miRNA molecules in the sample and the standard. Many of the analytical techniques discussed above can be used alone or in combination to provide information about the mRNA or miRNA content (including presence, absence, and/or relative abundance information) for comparison to a biomarker standard. For example, the DASL® assay can be used to establish a mRNA or miRNA profile for a sample from a subject and the abundances of specific identified molecules can be compared to the abundances of the same molecules in the biomarker standard.

Optionally, the detecting step comprises detecting the protein expression levels of the protein-coding gene biomarkers. The protein-coding gene biomarkers can comprise FOXO1A, SOX9, CLNS1A, PTGDS, XPO1, LETMD1, RAD23B, ABCC3, APC, CHES1, EDNRA, FRZB, HSPG2, and TMPRSS2_ETV1 FUSION. The protein detection can, for example, comprise an assay selected from the group consisting of Western blot, enzyme-linked immunosorbent assay (ELISA), enzyme immunoassay (EIA), radioimmunoassay (RIA), immunohistochemistry, and protein array. The analytical techniques used to determine protein expression are known. See, e.g., Sambrook et al., Molecular Cloning: A Laboratory Manual, 3^{rd} Ed., Cold Spring Harbor Press, Cold Spring Harbor, N.Y. (2001).

Biomarker standards can be predetermined, determined concurrently, or determined after a sample is obtained from the subject. Biomarker standards for use with the methods described herein can, for example, include data from samples from subjects without prostate cancer, data from samples from subjects with prostate cancer that is not a progressive, recurrent, and/or metastatic prostate cancer, and data from samples from subjects with prostate cancer that is a progressive, recurrent, and/or metastatic prostate cancer. Comparisons can be made to multiple biomarker standards. The standards can be run in the same assay or can be known standards from a previous assay.

Also provided herein are methods of treating a subject with prostate cancer. The methods comprise modifying a treatment regimen of the subject based on the results of any of the methods of predicting the recurrence, progression, and metastatic potential of a prostate cancer in a subject. Optionally, the treatment regimen is modified to be aggressive based on an increase in one or more biomarkers selected from the group consisting of CLNS1A, XPO1, LETMD1, RAD23B, TMPRSS2_ETV1 FUSION, ABCC3, APC, CHES1, FRZB, HSPG2, miR-103, miR-339, miR-183 and miR-182 as compared to a standard. Optionally, the treatment regimen is modified to be aggressive based on a decrease in one or more biomarkers selected from the group consisting of FOXO1A, SOX9, PTGDS, EDNRA, miR-136, and miR-221 as compared to a standard. Optionally, the treatment regimen is modified to be aggressive based on a combination of an increase in one or more biomarkers selected from the group consisting of CLNS1A, XPO1, LETMD1, RAD23B, TMPRSS2_ETV1 FUSION, ABCC3, APC, CHES1, FRZB, HSPG2, miR-103, miR-339, miR-183, and miR-182 and a decrease in one or more biomarkers selected from the group consisting of FOXO1A, SOX9, PTGDS, EDNRA, miR-136, and miR-221 as compared to a standard.

Also provided are methods of predicting recurrence potential of a disease. The methods comprise detecting gene expression profiles in subjects with the disease; detecting sets of clinical variables associated with the disease in the subjects with the disease; parametrically modeling the gene expression profiles and non-parametrically modeling the sets of clinical variables; and selecting gene expression profiles and sets of clinical variables consistent with a selected recurrence potential, wherein the selection step comprises Lasso type estimation. Optionally, the Lasso type estimation comprises a partly linear accelerated failure time model. Optionally, the disease is cancer. The cancer can, for example, be prostate cancer. Optionally, the gene expression profile is limited to a subset of genes. The subset of genes can, for example, comprise one or more genes selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

As used herein, parametrically modeling refers to modeling a family of distributions which can be described using a finite number of parameters. An example of a parametric model for outcome T and predictors Z is: Tᵢ=θ₁Zᵢ⁽¹⁾+L+θ_{d}Zᵢ^{(d)}+εᵢ, where θ are unknown parameters and d is a finite-dimensional.

As used herein non-parametrically modeling refers to modeling where the interpretation does not depend on fitting any parameterized distributions. Non-parametric models are widely used for studying populations that take on a ranked order (e.g., the Gleason score associated with prostate cancer). An example of a non-parametric model for outcome T and predictors X is: Tᵢ=φ(Xᵢ)+εᵢ, where φ is an unknown function to be estimated (i.e., an infinite dimensional parameter).

As used herein a lasso type estimation refers to the minimization of a convex loss function subject to L1-norm constraints on a finite number of unknown parameters in the loss function.

An example of a partly linear model is the following expression: Tᵢ=φ(Xᵢ)+ν₁Zᵢ⁽¹⁾+ . . . +ν_{d}Zᵢ^{(d)}+εᵢ, where T is a lifetime variable of interest, possibly right-censored and the unknown parameters are described above in the context of parametric models and non-parametric models.

Also provided are computer systems for predicting recurrence potential of a disease. The computer systems comprise a memory on which is stored a database comprising (i) a plurality of gene expression profiles for the disease, wherein each gene expression profile comprises a plurality of values, each value representing the expression level of a gene; (ii) a plurality of sets of clinical variables associated with the disease; and (iii) a descriptor associated with recurrence potential of the disease, wherein the descriptor is based on a combination of the gene expression profile and the set of clinical variables; and (b) a processor having computer-executable code for effecting the following steps: (i) parametrically modeling the gene expression profiles and non-parametrically modeling the sets of clinical variables; (ii) selecting gene expression profiles and sets of clinical variables consistent with a reference recurrence potential; and (iii) outputting the descriptor stating the recurrence potential for the disease based on the combination of the gene expression profile and the set of clinical variables. Optionally, the disease is a cancer. The cancer can, for example, be prostate cancer. Optionally, the gene expression profile is limited to a subset of genes. The subset of genes can, for example, comprise one or more genes selected from the group consisting of FOXO1A, SOX9, CLNS1A, PTGDS, XPO1, LETMD1, RAD23B, ABCC3, APC, CHES1, EDNRA, FRZB, HSPG2, TMPRSS_ETV1 FUSION, miR-103, miR-339, miR-183, miR-182, miR-136, and miR-221.

Also provided are kits comprising primers to detect the expression of biomarkers selected from the group consisting of FOXO1A, SOX9, CLNS1A, PTGDS, XPO1, LETMD1, RAD23B, and TMPRSS2_ETV1, and primers to detect the expression of biomarkers selected from the group consisting of miR-103, miR-339, miR-183, miR-182, miR-136, and miR-221. Optionally, directions to use the primers provided in the kit to predict the progression and metastatic potential of prostate cancer in a subject, materials needed to obtain RNA in a sample from a subject, containers for the primers, or reaction vessels are included in the kit.

Also provided are arrays consisting of probes to one or more of the biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRSS2_ETV1 FUSION, miR-103, miR-339, miR-183, miR-182, miR-136, and miR-221.

The arrays provided herein can be a DNA microarray, an RNA microarray, a miRNA microarray, or an antibody array. Arrays are known in the art. See, e.g., Dufva, Methods Mol. Biol. 529:1-22 (2009); Plomin and Schalkwyk, Dev. Sci. 10:1):19-23 (2007); Kopf and Zharhary, Int. J. Biochem. Cell Biol. 39(7-8):1305-17 (2007); Haab, Curr. Opin. Biotechnol. 17(4):415-21(2006); Thomson et al., Nat. Methods 1:47-53 (2004).

As used herein, subject can be a vertebrate, more specifically a mammal (e.g., a human, horse, cat, dog, cow, pig, sheep, goat mouse, rabbit, rat, and guinea pig), birds, reptiles, amphibians, fish, and any other animal. The term does not denote a particular age. Thus, adult and newborn subjects are intended to be covered. As used herein, patient or subject may be used interchangeably and can refer to a subject afflicted with a disease or disorder (e.g., prostate cancer). The term patient or subject includes human and veterinary subjects.

As used herein a subject at risk for recurrence, progression, or metastasis of prostate cancer refers to a subject who currently has prostate cancer, a subject who previously has had prostate cancer, or a subject at risk of developing prostate cancer. A subject at risk of developing prostate cancer can be genetically predisposed to prostate cancer, e.g., a family history or have a mutation in a gene that causes prostate cancer. Alternatively a subject at risk of developing prostate cancer can show early signs or symptoms of prostate cancer, such as hyperplasia. A subject currently with prostate cancer has one or more of the symptoms of the disease and may have been diagnosed with prostate cancer.

As used herein, the terms treatment, treat, or treating refers to a method of reducing the effects of a disease or condition (e.g., prostate cancer) or symptom of the disease or condition. Thus, in the disclosed method, treatment can refer to a 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### General Methods

### RNA Isolation

RNA is isolated from formalin-fixed paraffin-embedded (FFPE) tissue according to the methods described in Abramovitz et al., Biotechniques 44(3):417-23 (2008). In brief, three 5 μm sections per block were cut and placed into a 1.5 mL sterile microfuge tube. The tissue section was deparaffinized with 100% xylene for 3 minutes at 50° C. The tissue section was centrifuged, washed twice with ethanol, and allowed to air dry. The tissue section was digested with Proteinase K for 24 hours at 50° C. RNA was isolated using an Ambion Recover All Kit (Ambion; Austin, Tex.).

cDNA-Mediated Annealing, Selection, Extension, And Ligation Assay (DASL® Assay)

Upon the completion of RNA isolation, the isolated RNA is used in the DASL® assay. The DASL® assay is performed according to the protocols supplied by the manufacturer (Illumina, Inc.; San Diego, Calif.). The primer sequences for the fourteen biomarker genes are shown in Table 1. The probe sequences for the fourteen biomarker genes are shown in Table 2.

TABLE 1
DASL ® assay Primer Sequences for Fourteen
Biomarker Genes

| Gene | Primer Sequences |
|---|---|
| FOXO1A | 5′-ACTTCGTCAGTAACGGACGTCCTAGGAGAAGAG |
| | CTGCATCCA-3′ |
| | (SEQ ID NO: 1) |
| | 5′-GAGTCGAGGTCATATCGTGTCCTAGGAGAAGAG |
| | CTGCATCCA-3′ |
| | (SEQ ID NO: 2) |
| SOX9 | 5′-ACTTCGTCAGTAACGGACGCTCCTACCCGCCCA |
| | TCACCC-3′ |
| | (SEQ ID NO: 3) |
| | 5′-GAGTCGAGGTCATATCGTGCTCCTACCCGCCCA |
| | TCACCC-3′ |
| | (SEQ ID NO: 4) |
| CLNS1A | 5′-ACTTCGTCAGTAACGGACGGAGAGAACTTGGTG |
| | CCTCTTCC-3′ |
| | (SEQ ID NO: 5) |
| | 5′-GAGTCGAGGTCATATCGTGGAGAGAACTTGGTG |
| | CCTCTTCC-3′ |
| | (SEQ ID NO: 6) |
| PTGDS | 5′-ACTTCGTCAGTAACGGACGCGAACCCAGACCCC |
| | CAGG-3′ |
| | (SEQ ID NO: 7) |
| | 5′-GAGTCGAGGTCATATCGTGCGAACCCAGACCCC |
| | CAGG-3′ |
| | (SEQ ID NO: 8) |
| XPO1 | 5′-ACTTCGTCAGTAACGGACGCCAGCAAAGAATGG |
| | CTCAAGAA-3′ |
| | (SEQ ID NO: 9) |
| | 5′-GAGTCGAGGTCATATCGTGCCAGCAAAGAATGG |
| | CTCAAGAA-3′ |
| | (SEQ ID NO: 10) |
| LETMD1 | 5′-ACTTCGTCAGTAACGGACGTCACCTTTCTCCAA |
| | AGGCAGATG-3′ |
| | (SEQ ID NO: 11) |
| | 5′-GAGTCGAGGTCATATCGTGTCACCTTTCTCCAA |
| | AGGCAGATG-3′ |
| | (SEQ ID NO: 12) |
| RAD23B | 5′-ACTTCGTCAGTAACGGACAATCCTTCCTTGCTT |
| | CCAGCG-3′ |
| | (SEQ ID NO: 13) |
| | 5′-GAGTCGAGGTCATATCGTAATCCTTCCTTGCTT |
| | CCAGCG-3′ |
| | (SEQ ID NO: 14) |
| TMPRSS2_ETV1 | 5′-ACTTCGTCAGTAACGGACAGCGCGGCACTCAGG |
| FUSION | TACCT-3′ |
| | (SEQ ID NO: 15) |
| | 5′-ACTTCGTCAGTAACGGACAGCGCGGCACTCAGG |
| | TACCT-3′ |
| | (SEQ ID NO: 16) |
| ABCC3 | 5′-ACTTCGTCAGTAACGGACATGTTCCTGTGCTCC |
| | ATGATGC-3′ |
| | (SEQ ID NO: 17) |
| | 5′-GAGTCGAGGTCATATCGTATGTTCCTGTGCTCC |
| | ATGATGC-3′ |
| | (SEQ ID NO: 18) |
| | 5′-GTCGCTGATCTTACAACACTATTACATGCCTAT |
| | TGACGTGAGGCGGTCTGCCTATAGTGAGTC-3′ |
| | (SEQ ID NO: 19) |
| APC | 5′-ACTTCGTCAGTAACGGACGTCCCTGGAGTAAAA |
| | CTGCGGTC-3′ |
| | (SEQ ID NO: 20) |
| | 5′-GAGTCGAGGTCATATCGTGTCCCTGGAGTAAAA |
| | CTGCGGTC-3′ |
| | (SEQ ID NO: 21) |
| | 5′-AAAATGTCCCTCCGTTCTTATCTAGATCGCAAA |
| | AGTGTCTCGGAAGTCTGCCTATAGTGAGTC-3′ |
| | (SEQ ID NO: 22) |
| CHES1 | 5′-ACTTCGTCAGTAACGGACGGGTTTCTCCAAGGC |
| | CCTTCA-3′ |
| | (SEQ ID NO: 23) |
| | 5′-GAGTCGAGGTCATATCGTGGGTTTCTCCAAGGC |
| | CCTTCA-3′ |
| | (SEQ ID NO: 24) |
| | 5′-GAAGACGATGACCTCGACTTCATACGCGAATTG |
| | ATAGAAGCTCGGTCTGCCTATAGTGAGTC-3′ |
| | (SEQ ID NO: 25) |
| EDNRA | 5′-ACTTCGTCAGTAACGGACTGCAACTCTGCTCAG |
| | GATCATTT-3′ |
| | (SEQ ID NO: 26) |
| | 5′-GAGTCGAGGTCATATCGTTGCAACTCTGCTCAG |
| | GATCATTT-3′ |
| | (SEQ ID NO: 27) |
| | 5′-CCAGAACAAATGTATGAGGAATTCACTCAAGGC |
| | CGTTAGCTGTGGTCTGCCTATAGTGAGTC-3′ |
| | (SEQ ID NO: 28) |
| FRZB | 5′-ACTTCGTCAGTAACGGACGGAAGCTTCGTCATC |
| | TTGGACTCAG-3′ |
| | (SEQ ID NO: 29) |
| | 5′-GAGTCGAGGTCATATCGTGGAAGCTTCGTCATC |
| | TTGGACTCAG-3′ |
| | (SEQ ID NO: 30) |
| | 5′-AAAAGTGATTCTAGCAATAGTGATTTTACTGCG |
| | CTCCTAATTGGCACCGTCTGCCTATAGTGAGTC-3′ |
| | (SEQ ID NO: 31) |
| HSPG2 | 5′-ACTTCGTCAGTAACGGACCCAAATGCGCTGGAC |
| | ACATT-3′ |
| | (SEQ ID NO: 32) |
| | 5′-GAGTCGAGGTCATATCGTCCAAATGCGCTGGAC |
| | ACATT-3′ |
| | (SEQ ID NO: 33) |
| | 5′-GTACCTTTCTGATGATGAGGACGGAACAGCTTA |
| | CGACTTTGCGGGTCTGCCTATAGTGAGTC-3′ |
| | (SEQ ID NO: 34) |

TABLE 2
Probe Sequences for Detection of Fourteen
Biomarker Genes in DASL ® assay

| Gene | Probe Sequence |
|---|---|
| FOXO1A | 5′-TCCTAGGAGAAGAGCTGCATCCATGGACAACAA |
| | CAGTAAATTTGCTA-3′ |
| | (SEQ ID NO: 35) |
| SOX9 | 5′-CTCCTACCCGCCCATCACCCGCTCACAGTACGA |
| | CTACACCGAC-3′ |
| | (SEQ ID NO: 36) |
| CLNS1A | 5′-GGAGAGAACTTGGTGCCTCTTCCACTCTGGAGT |
| | GAAGTTAATGAAAG-3′ |
| | (SEQ ID NO: 37) |
| PTGDS | 5′-CGAACCCAGACCCCCAGGGCTGAGTTAAAGGAG |
| | AAATTCACC-3′ |
| | (SEQ ID NO: 38) |
| XPO1 | 5′-CCAGCAAAGAATGGCTCAAGAAGTACTGACACA |
| | TTTAAAGGAGCAT-3′ |
| | (SEQ ID NO: 39) |
| LETMD1 | 5′-TCACCTTTCTCCAAAGGCAGATGTGAAGAACTT |
| | GATGTCTTATGTGG-3′ |
| | (SEQ ID NO: 40) |
| RAD23B | 5′-AATCCTTCCTTGCTTCCAGCGTTACTACAGCAG |
| | ATAGGTCGAGAG-3′ |
| | (SEQ ID NO: 41) |
| TMPRSS2_ETV1 | 5′-AGCGCGGCACTCAGGTACCTGACAATGATGAGC |
| FUSION | AGTTTGTACC-3′ |
| | (SEQ ID NO: 42) |
| ABCC3 | 5′-ATGTTCCTGTGCTCCATGATGCAGTCGCTGATC |
| | TTACAACACTATT-3′ |
| | (SEQ ID NO: 43) |
| APC | 5′-TCCCTGGAGTAAAACTGCGGTCAAAAATGTCCC |
| | TCCGTTCTTAT-3′ |
| | (SEQ ID NO: 44) |
| CHES1 | 5′-GGTTTCTCCAAGGCCCTTCAGGAAGACGATGAC |
| | CTCGACTT-3′ |
| | (SEQ ID NO: 45) |
| EDRNA | 5′-TGCAACTCTGCTCAGGATCATTTACCAGAACAA |
| | ATGTATGAGGAAT-3′ |
| | (SEQ ID NO: 46) |
| FRZB | 5′-GAAGCTTCGTCATCTTGGACTCAGTAAAAGTGA |
| | TTCTAGCAATAGTGATT-3′ |
| | (SEQ ID NO: 47) |
| HSPG2 | 5′-CCAAATGCGCTGGACACATTCGTACCTTTCTGA |
| | TGATGAGGAC-3′ |
| | (SEQ ID NO: 48) |

To compute the predictive fourteen-gene score, DASL® signal levels are quantile normalized across the array, and then Z-score normalized across the samples. (Z-score=(signal−average(signal))/stdev(signal)). Once the predictive scores are computed, samples are separated based on whether they are greater or less than the median score. If a sample has a score greater than the median, the subject is predicted to not have recurrence. If the score is less than the median, the subject is predicted to have recurrence. For this predictive score, the higher the score, the less likely the subject is to have recurrence.

The predictive fourteen-gene score can be calculated using the following formula:

FOURTEEN GENE SCORE=(C_{FOXO1A}×FOXO1A_{Zscore})+(C_{SOX9}×SOX9_{Zscore})+(C_{CLNS1A}×CLNS1A_{Zscore})+(C_{PTGDS}×PTGDS_{Zscore})+(C_{XPO1}×XPO1_{Zscore})+(C_{RAD23B}×RAD23B_{Zscore})+(C_{TMPRSS2}_{_{—}}_{ETV1 FUSION}×TMPRSS2_{—}EVT1FUSION_{Zscore})+(C_{ABCC3}×ABCC3_{Zscore})+(C_{APC}×APC_{Zscore})=(C_{CHES1}×CHES1_{Zscore})+(C_{EDNRA}×EDNRA_{Zscore})+(C_{FRZB}×FRZB_{Zscore})+(C_{HSPG2}×HSPG2_{Zscore}).

The coefficients for the predictive fourteen-gene score are as follows:

C_{FOXO1A}=0.687, C_{SOX9}=0.351, C_{CLNS1A}=0.112, C_{PTGDS}=0.058, C_{XPO1}=−0.208, C_{LETMD1}=−0.019, C_{RAD23B}=−0.065, C_{TMPRSS2}_{_{—}}_{ETV1 FUSION}=−0.168, C_{ABCC3}=−0.202, C_{APC}=−0.128, C_{FRZB}=0.310, C_{HSPG2}=−0.048, C_{EDNRA}=0.539, and C_{CHES1}=−0.143.

The coefficients for the predictive seven-gene score are as follows: C_{FOXO1A}=0.625, C_{SOX9}=0.253, C_{CLNS1A}=0.0, C_{PTGDS}=0.056, C_{XPO1}=−0.092, C_{LETMD1}=−0.140, C_{RAD23B}=−0.045, and C_{TMPRSS2}_{_{—}}_{ETV1 FUSION}=−0.137.

miRNA Expression Profiling

The isolated RNA is additionally used in the Illumina Human Version 2 MicroRNA Expression Profiling kit (Illumina, Inc.; San Diego, Calif.) in conjunction with the DASL® assay. The miRNA expression profiling is performed according to the manufacturer's protocol. The mature miRNA sequence for the six miRNA biomarkers are shown in Table 3. The probe sequences for the six miRNA biomarkers are shown in Table 4.

TABLE 3
Mature miRNA Sequences for Six miRNA Biomarkers

| Gene | Mature miRNA sequence |
|---|---|
| Hsa-miR-103 | 5′-AGCAGCATTGTACAGGGCTATGA-3′ |
| | (SEQ ID NO: 49) |
| Hsa-miR-339 | 5′-TCCCTGTCCTCCAGGAGCTCA-3′ |
| | (SEQ ID NO: 50) |
| Hsa-miR-183 | 5′-TATGGCACTGGTAGAATTCACTG-3′ |
| | (SEQ ID NO: 51) |
| Hsa-miR-182 | 5′-TTTGGCAATGGTAGAACTCACA-3′ |
| | (SEQ ID NO: 52) |
| Hsa-miR-136 | 5′-AGCTACATTGTCTGCTGGGTTTC-3′ |
| | (SEQ ID NO: 53) |
| Hsa-miR-221 | 5′-ACTCCATTTGTTTTGATGATGGA-3′ |
| | (SEQ ID NO: 54) |

TABLE 4
Probe Sequences for Detection of Six miRNA
Biomarker Genes in DASL ® assay

| Gene | Probe Sequence |
|---|---|
| Hsa-miR-103 | 5′-ACTTCGTCAGTAACGGACTCCAGTAGCGACTA |
| | GCCCGTCAGCAGCATTGTACAGGGCTA-3′ |
| | (SEQ ID NO: 55) |
| Hsa-miR-339 | 5′-ACTTCGTCAGTAACGGACTATACCGGCCTAAG |
| | CACTCGCACCCTGTCCTCCAGGAGCT-3′ |
| | (SEQ ID NO: 56) |
| Hsa-miR-183 | 5′-ACTTCGTCAGTAACGGACAATGTTGACCCGGAT |
| | CTCGTCCATGGCACTGGTAGAATTCA-3′ |
| | (SEQ ID NO: 57) |
| Hsa-miR-182 | 5′-ACTTCGTCAGTAACGGACACTAGCCCTCGCATA |
| | GCTTGCGTTTGGCAATGGTAGAACTC-3′ |
| | (SEQ ID NO: 58) |
| Hsa-miR-136 | 5′-ACTTCGTCAGTAACGGACGCGCAATTCCCTCGA |
| | TCTTACGCTACATTGTCTGCTGGGT-3′ |
| | (SEQ ID NO: 59) |
| Hsa-miR-221 | 5′-ACTTCGTCAGTAACGGACGTAGGTCCCGGACGT |
| | AATCACCACTCCATTTGTTTTGATGAT-3′ |
| | (SEQ ID NO: 60) |

To compute a predictive miRNA score, DASL signal levels are quantile normalized across the array, and then Z-score normalized across the samples. (Z-score=(signal−average(signal))/stdev(signal)). The more positive the predictive score, the more likely the subject will recur. The more negative the score, the less likely the patient will recur.

The predictive six miRNA gene score can be calculated using the following formula:

SIX miRNA SCORE=miR-103_{Zscore}+miR-339_{Zscore}+miR-183_{Zscore}+miR-182_{Zscore}−miR136_{Zscore}−miR221_{Zscore.}

### Example 1

### Identification of Biomarker Predictors for the Progression and Metastatic Potential of Prostate Cancer

A highly predictive set of 520 genes was determined through analysis of multiple publicly available gene expression datasets (Dhanasekaran et al., Nature 412:822-6 (2001); Lapointe et al., Proc. Natl. Acad. Sci. USA 101:811-6 (2004); LaTulippe et al., Cancer Res. 62:4499-506 (2002); Varambally et al., Cancer Cell 8:393-406 (2005)), datasets from gene expression profiling analysis of 58 prostate cancer patient samples (Liu et al., Cancer Res. 66:4011-9 (2006)), and genes involved in prostate cancer progression based on state of the art understanding of the disease (Tomlins et al., Science 310:644-8 (2005); Varambally et al., Cancer Cell 8:393-406 (2005)). The predictive set of 520 genes were optimized for performance in the cDNA-mediated annealing, selection, extension, and ligation (DASL®) assay (Illumina, Inc.; San Diego, Calif.). The DASL® assay is based upon multiplexed reverse transcription-polymerase chain reaction (RT-PCR) applied in a microarray format and enables the quantitation of expression of up to 1536 probes using RNA isolated from archived formalin-fixed paraffin embedded (FFPE) tumor tissue samples in a high throughput format (Bibokova et al., Am. J. Pathol. 165:1799-807 (2004); Fan et al., Genome Res. 14:878-85 (2004)). RNA was isolated from 71 patient samples with definitive clinical outcomes and was analyzed using the DASL® assay. Based on the data from 71 patients, a subset of fourteen protein encoding genes were found to be capable of separating Gleason 7 subjects with and without recurrence, and thus were found to be good predictors of recurrent, progressive, or metastatic prostate cancers. The fourteen protein encoding genes included: FOXO1A, SOX9, CLNS1A, PTGDS, XPO1, LETMD1, RAD23B, ABCC3, APC, CHES1, EDNRA, FRZB, HSPG2, and the TMPRSS2_ETV1 FUSION. The expression of CLNS1A, XPO1, LETMD1, RAD23B, ABCC3, APC, CHES1, FRZB, HSPG2, and TMPRSS2_ETV1 FUSION was increased in recurrent, progressive, or metastatic prostate cancers, while the expression of FOXO1A, SOX9, EDNRA, and PTGDS was decreased in recurrent, progressive, or metastatic prostate cancers. Additionally, based on data obtained from the 71 patients using the MicroRNA Expression Profiling Panels (Illumin, Inc.; San Diego, Calif.) designed for the DASL® assay, it was found that six miRNA genes were found to be good predictors of recurrent, progressive, or metastatic prostate cancers. The six miRNA genes included: miR-103, miR-339, miR-183, miR-182, miR-136, and miR-221. The expression of miR-103, miR-339, miR-183, and miR-182 was increased in recurrent, progressive, or metastatic prostate cancers, while the expression of miR-136 and miR-221 was decreased in recurrent, progressive, or metastatic prostate cancers.

### Example 2

### Determination of Novel Partly Linear Accelerated Failure Time (AFT) Model

### Feature Selection in AFT

The accelerated failure time (AFT) model is an important tool for the analysis of censored outcome data (Cox and Oakes, Analysis of Survival Data, Chapman and Hall, London, England (1984); Kalbfleisch and Prentice, The Statistical Analysis of Failure Tie Data, John Wiley, New York, N.Y. (2002)). Classic AFT models assume that the covariate effects on the logarithm of the time-to-event are linear, in which case standard rank-based techniques for estimation and inference could be used (Jin et al., Biometrika 90:341-53 (2003)), and its extension for lasso-type regularized variable selection could be considered (Cai et al., Biometrics, In press, 2009). Regarding these variable selection procedures, there are two unsatisfying products. First, it is assumed that the clinical effects are linear. Second, an unsupervised implementation of the regularized variable selection procedure can inadvertently remove clinical variables that are known to be scientifically relevant and can be measured easily in practice. While the second limitation can be addressed by tweaking the underlying estimation scheme, the first limitation remains. Alternatively, many authors ignore important clinical covariate effects when selecting important gene features; here, those important clinical covariate effects were considered. Both concerns in the context of AFT models were addressed.

### Partly Linear Models

Linear regression functions are insufficient in many applications, and it is more desirable to allow for more general covariate effects. The nonparametric modeling of covariate effects is less restrictive than the parametric approach, and thus, is less likely to distort the underlying relationship between the failure time and the covariate. However, new challenges arise when including nonlinear covariate effects in regression models. For instance, it is known that nonparametric repression methods may encounter the so-called curse of dimensionality problem, when the dimension of covariates is high, e.g., a large number of gene biomarkers are used. The partly linear model of Engle et al. provides a useful compromise to model the effect of some covariates nonparametricaly and the rest parametrically (Engle et al., J. Am. Stat. Assoc. 81:310-20 (1986)). The partly linear model is a ubiquitous concept in the statistics literature and an important tool in modern semiparametric regression (Hardle et al., Partially Linear Models, Springer, New York, N.Y. (2000); Ruppert et al., Semiparametric Regression, Cambridge University Press, New York, N.Y. (2002)). Specifically, for the i-th subject, let Tᵢ be a univariate endpoint of interest for the i-th subject, and Zᵢ (d×1) and Xᵢ (q×1) denotes features of interest (e.g., gene expression levels) and clinical variables, respectively. Then one partly linear model is:

Tᵢ=φ(Xᵢ⁽¹⁾, . . . ,Xᵢ^{(q)})+νZᵢ⁽¹⁾+ . . . +νZᵢ^{(d)}+εᵢ, (1)

where ν=(ν₁, . . . , ν_{d})^{T} is a parameter vector of interest, φ is an unspecified function, and the errors are i.i.d. and follow an arbitrary distribution function F_{ε}. Special cases of this model have been used in varied applications across many disciplines including econometrics, engineering, biostatistics, and epidemiology (Hardle et al., Partially Linear Models, Springer, New York, N.Y. (2000)). Estimation and inference for when the outcomes Ti may be right-censored were considered herein, in which case the observed data is {(\check{T}ᵢ, δᵢ, Zᵢ, Xᵢ)}ⁿᵢ₌₁, where \check{T}ᵢ=min (Tᵢ, Cᵢ), di=I(Tᵢ≦Cᵢ), Cᵢ is a random censoring event, Zᵢ=(Zᵢ⁽¹⁾, . . . , Zᵢ^{(d)})^{T}, and Xᵢ=(Xᵢ⁽¹⁾, . . . , Xᵢ^{(q)})^{T}. In the context of survival analysis, Tᵢ is the log-transformed survival time, and Model (1) is referred to as partly linear AFT models.

In the absence of censoring, the nonparametric function φ in Model (1) can be estimated using kernel methods, especially when q=1 (Hardle et al., Partially Linear Models, Springer, New York, N.Y. (2000) and references therein) and smoothing spline methods (Engle et al., J. Am. Stat. Assoc. 81:310-20 (1986); Heckman, J. Royal Stat. Soc. Series B 48:244-8 (1986); Green and Silverman, Nonparametric Regression and Generalized Linear Models: a Roughness Penalty Approach, Chapman and Hall, New York, N.Y. (1994)). To extend the partly linear models in the context of AFT models, one approach is to extend the basic weighting scheme of Koul et al. (Koul et al., Annals of Stat. 9:1276-88 (2006)). Here censoring is treated like other missing data problems (Tsiatis, Ann. Statist. 18:354-72 (1990)) and inversely weights the uncensored observations by the probability of being uncensored, i.e., so-called inverse-probability weighted (IPW) estimators. A close cousin of to the IPW methodology is censoring unbiased transformations (Fan and Gijbels, Local Polynomial Modeling and Its Applications, Chapter 5 and references therein, Chapman and Hall, New York, N.Y. (1996)), which effectively aims to replace censored outcome with a suitable surrogate. After the transformation is made, complete-data estimation procedures can be applied. Both IPW kernel-type estimators and censoring unbiased transformation in the partly linear model have been studied for AFT models (Liang and Zhou, Comm. Statist. Theory Method 27:2895-907 (1998); Wang and Li, J. Multivariate Anal. 83:469-86 (2002)).

A general penalized loss function for partly linear AFT models is considered herein:

$${min}_{β , {φ}_{ɛΦ}} {L}_{n} ( φ , υ ) + λ J ( φ ) , \qquad ( 2 )$$

where Lₙ is the loss function for observed data and J(φ) imposes some type of penalty on the complexity of φ. The approach will be to replace Lₙ with the Gehan (Genhan, Biometirka 52:203-23 (1965)) loss function (Jin et al., Biometrika 90:341-53 (2003)) and model φ using penalized regression splines. Variable selection and building predictive scores as well as estimation for the regression parameter u, is described herein. To minimize the penalized loss function (Model (2)), the insight into the optimization procedure is due, in part, to Koenker et al., which noted that the optimization problem in quantile smoothing splines can be solved by L₁-type linear programming techniques. Subsequently, an interior point algorithm was proposed for the problem. (Koenker et al., Biometrika 81:673-80 (1994)). Li et al., build on this idea to propose an entirely different path-finding algorithm to replace the interior point algorithm of Koenker et al. (Li et al., J. Am. Stat. Assoc. 102:255-68 (2007); Koenker et al., Biometrika 81:673-80 (1994)). In a related paper, Li and Zhu adopt a similar approach for lasso-type variable selection in quantile regression (Li and Zhou, J. Comp. Graph. Stat. 17:163-185 (2008); Tibshirani, J. Roy. Stat. Soc., Ser. B 58:267-88 (1996)). Following the work of Koenker et al. (1994), it can be readily shown when J(φ) is taken as a L₁ norm as discussed in penalized regression spline literature, there is a close connection to between our loss function (2) and the lasso-type problem; specifically, the optimization problem of (2) is essentially an L₁ loss plus L₁ penalty problem. This statement is true regardless of the algorithm, and this relation was exploited in the approach to the optimization problem. Once the basic spline framework is adopted, it was shown that the estimator can be generalized through additive models for q>1 and variable selection in the parametric component. The additive structure of the nonparametric components is adopted to further alleviate the issue of curse of dimensionality, when q>1 (Hastie and Tibshirani, Generalized Additive Models, Chapman and Hall, New York, N.Y. (1990)).

### Regression Splines in Partly Linear AFT Model

A simplified case for the partly linear model was first considered, where Xᵢ is assumed to be univariate, i.e., q=1 and Xᵢ≡Xᵢ, and then Model (1) reduces to:

Tᵢ=φ(Xᵢ)+ν₁Zᵢ⁽¹⁾+ . . . +ν_{d}Zᵢ^{(d)}+εᵢ, (3)

The model in (3) agrees with the model considered by Chen et al. (Chen et al., Statistica Sinica 15:767079 (2005)). Let B(x)={B₁(x), . . . , B_{M}(x)}^{T}, M≦n, be a set of linearly independent piecewise polynomial basis functions. The piecewise polynomial model asserts that φ(x)=B(x)^{T}β, for some β, βεR^{M} and E(εᵢ)=α. Popular bases are B-splines, natural splines, and truncated power series basis (Ruppert et al., Semiparametric Regression, Cambridge University Press, New York, N.Y. (2002)). The truncated power series basis of degree p without the intercept term was chosen, that is, B(x)={x, . . . , x^{p}, (x−κ₁)^{p}₊, . . . , (x−κᵣ)^{p}₊}^{T}, where (κ₁, . . . , κᵣ) denotes the knots, r≧1 is the number of knots and (u)₊=uI(u≧0), and hence M=p+r. Throughout, equally spaced percentiles were used as knots and set p=3, i.e., the cubic splines, unless otherwise noted. Then define

$$\hat{θ} ≡ ( \hat{β} , \hat{ϑ} ) = {min}_{β , ϑ} {ℒ}_{n} ( β , ϑ ) , \\ where \\ {ℒ}_{n} ( β , ϑ ) = {n}^{- 2} {\sum }_{i = 1}^{n} {\sum }_{j = 1}^{n} {δ}_{i} ( {e}_{i} - {e}_{j} ) \_ , \qquad ( 4 )$$

with eᵢ=\check{T}i−β^{T}B(Xᵢ)−ν^{T}Zᵢ. Because the model (3) has been “linearized,” existing rank-based estimation techniques can be applied for the usual AFT models. In particular, it is noted that the minimizer Lₙ(β, ν) is also the minimizer of

$${\sum }_{i = 1}^{n} {\sum }_{j = 1}^{n} {δ}_{i} \vert {e}_{i} - {e}_{j} \vert + \vert ζ - ( {β}^{T} , {ϑ}^{T} ) {\sum }_{k = 1}^{n} {\sum }_{l = 1}^{n} {δ}_{k} {D}_{lk} \vert ,$$

for a large constant ζ, where Dₗₖ={B(Xₗ)^{T}, Z^{T}ₗ}^{T}−{B(Xₖ)^{T}, Z^{T}ₖ}^{T}. (Jin et al., Biometrika 90:341-53 (2003)). Evidently, the minimizer of the new loss function may be viewed as the solution to the least absolute deviation (lad) regression of a pseudo response vector V=(V₁, . . . , Vₛ)^{T} (S×1) on a pseudo design matrix W=(W₁, . . . , W_{S})^{T} (S×(M+d)). It can readily be shown that the pseudo response vector V is of the form {δᵢ(\check{T}ᵢ−\check{T}ⱼ), . . . , ζ}^{T} and the pseudo design matrix W is of the form, where δᵢ(\check{T}ᵢ−\check{T}ⱼ) and δᵢD^{T}ᵢⱼ go through all i and j with δᵢ=1. Without loss of generality, write

$${\hat{θ}}_{RS} = min {\sum }_{s = 1}^{S} \vert {V}_{s} - {θ}^{T} {W}_{s} \vert . \qquad ( 5 )$$

The fact that θ_{RS} can be written as the lad regression estimate facilitates the estimation techniques for the model of interest presented below. The estimator θ_{RS} is a regression spline estimator where, for fixed knots, its root-n consistency and asymptotic normality can be established by extending previous work for linear AFT models (Tsiatis, Ann. Stat. 18:354-72 (1990); Jin et al., Biometrika 90:341-53 (2003)).

### Penalized Regression Splines in Partly Linear AFT Models

When regression splines are used to model nonparametric covariates effects, it is crucial to choose the optimal number and location of knots (κ₁, . . . , κᵣ). It is well known that too many knots may lead to overfitting whereas too few knots may not be sufficient to capture the non-linear effect. To conduct knots selection, various sophisticated procedures have been developed and can be used to improve the performance of the regression spline models. Among these procedures, the so-called penalized regression spline method (Eilers and Marz, Stat. Science 11:89-121(1996); Ruppert and Carroll, Unpublished Technical Report (1997)) is particularly attractive, which is simpler to implement and often enjoys better performance. For penalized regression spline models, a large number of knots can be included and overfitting is avoided through regularization, e.g., a L₁ type of penalty on the regression coefficients. This approach was adopted and the penalized regression spline estimator for the partly linear AFT model was considered

$${\hat{θ}}_{PRS} ( γ ) = {min}_{β , ϑ} \{ {ℒ}_{n} ( β , ϑ ) + γ {\sum }_{m = p + 1}^{M} \vert {β}_{m} \vert \} , \qquad ( 6 )$$

where M=p+r and γ is a regulation parameter on the jumps in the pth derivative and is used achieve the goal of knot selection. Using the lad loss function in (5) and a standard data augmentation technique for regularized lad regression, the penalized estimate may be found easily. Namely, define V*=(V^{T}, 0^{T}ᵣ)^{T}, W*=[W^{T}, (0_{r×p}, Dᵣ, 0_{r×d})^{T}]^{T}, and Dᵣ=γIᵣ, where 0ᵣ is a r-vector of zeros, 0_{r×p} (0_{r×d}) is a r×p (r×d) matrix of zeroes and Iᵣ an r-dimensional identity matrix. Then, θ_{PRS}_{_{(γ)}} is found through the lad regression of V* on W*. A penalized regression spline with L₁ penalty corresponds to a Bayesian model with double exponential or Laplace priors and is known to be able to accommodate large jumps (Ruppert and Carroll, Unpublished Technical Report (1997)).

### Variable Selection in Partly Linear AFT Models

Finally, variable selection was considered for Z=(Z^{T}₁, . . . , Z^{T}ₙ)^{T} (gene expression data) in partly linear AFT model (1) by extending the penalized regression spline estimator θ_{PRS}_{_{(γ)}}. Let λ=(λ₁, . . . , λ_{d}) be covariate-dependent regularization parameters and consider the minimizer to the convex loss function

$${\hat{θ}}_{PRS ( 1 )} ( γ , λ ) = {min}_{β , ϑ} \{ {ℒ}_{n} ( β , ϑ ) + γ {\sum }_{m = p + 1}^{M} \vert {β}_{m} \vert + λ {\sum }_{j = 1}^{d} \vert {ϑ}_{j} \vert \} . \qquad ( 7 )$$

The same data augmentation scheme used for regression splines and penalized regression splines applies to the lasso-type estimator (7) as well. Define the pseudo response vector V^{\}=(V^{T}, 0^{T}_{r+d})^{T} and the pseudo design matrix

$${W}^{†} = {[ {W}^{T} , {( \begin{matrix}{0}_{r \times p} & γ {I}_{r} & {0}_{r \times d} \\ {0}_{d \times p} & {0}_{d \times r} & diag ( {λ}_{1} , … , {λ}_{d} )\end{matrix} )}^{T} ]}^{T} .$$

For fixed γ and λ, the estimate is computed as the lad regression estimate of V^{\} on W^{\}. To select γ and λ, two approaches were proposed, namely the cross validation (CV) and generalized cross validation (GCV). The K-fold CV approach chooses the values of γ and λ that maximize the Gehan loss function (4). The GCV approach chooses the values for γ and λ that maximize the criteria, Ln (β, ν)/(1−d_{γ,λ}/n)², where n is the number of observations and d_{γ,λ} is the number of nonzero estimated coefficients for the basis functions B(x) and Z linear predictors, that is, the number of nonzero estimates in (β, ν). Note that d_{γ,λ} depends on γ and λ.

### Extension to Additive Partly Linear AFT Models

In the case where q is greater than 1 in the partly linear model (1), it is well-known that the estimation is difficult due to the issue of curse of dimensionality, even when q is moderate and it is in the absence of censoring. For the partly linear AFT model presented herein, an additive structure was proposed to be used for φ to further alleviate the problem, namely, an additive partly linear AFT model,

$${T}_{i} = {\sum }_{j = 1}^{q} {φ}_{j} ( {X}_{i}^{( j )} ) + {ϑ}_{1} {Z}_{i}^{( 1 )} + … + {ϑ}_{d} {Z}_{i}^{( d )} + {ɛ}_{i} , \qquad ( 8 )$$

where φⱼ's (j=1, . . . , q) are unknown functions and are estimated through regression splines. Penalized regression splines can be used for additive partly linear model to conduct knot selection for each nonparametric effect φⱼ(Xᵢ^{(j)})(j=1, . . . , q). The variable selection for Z can also be easily extended to this additive partly linear AFT model. When q is large and it is also of interest to conduct feature selection among the q nonparametrically modeled effects, one can modify the regularization term for β in the loss function (6) and (7); specifically, one can regularize all β, i.e., γΣ^{M}ₘ₌₁|βₘ|, as opposed to only regularize the terms that correspond to the set of jumps in the pth derivative, γΣ^{M}ₘ₌ₚ₊₁|βₘ|. Similarly, the data augmentation scheme to obtain the parameter estimates for these models can be modified.

### Example 3

### Simulation Studies

Multiple simulation studies were conducted to evaluate the operating characteristics of the methods in comparison with several other methods. All calculations were conducted in R and the models described herein were fit using the algorithms proposed above, which utilize the quantreg package in R.

### Estimation

A case of single covariate and single covariate Xᵢ in (1) was first considered and the estimates of the regression coefficient ν and its sampling variance were focused on. Note in this setup, no feature selection was involved. To facilitate comparisons, the simulation study details were adapted from those given by Chen et al. (Chen et al., Statistica Sinica 15:767-79 (2005)). It was assumed that the partly linear model (1) holds with ν=1 and εᵢ˜N(0, σ²) with σ²=1 and mutually independent of (Xᵢ, Zᵢ). The random variable Xᵢ was correlated with Zᵢ through the regression relation Xᵢ=0.25 Zᵢ+Uᵢ, where Uᵢ is Un(−5, 5) and completely independent of all other random variables. As in Chen et al., linear and quadratic effects were considered, φ(Xᵢ)=2 Xᵢ and φ(Xᵢ)=Xᵢ², respectively. Finally, censoring random variables were simulated according to the rule, Ci=φ(Xᵢ)+Zᵢν+Iᵢ* follows the uniform distribution Un (0, τ) with τ=1.6. As a result, the proportion of censored outcomes ranges from 20% to 30%. The estimator, the partly linear AFT model (PL-AFT) with r knots (r=2 and 4), which was fit using the loss function (6), was compared to the stratified estimator of Chen et al. (Chen et al., Statistica Sinica 15:767-79 (2005)) (S_{K}-AFT) where K denotes the number of strata, the usual AFT model with both Xᵢ and Zᵢ modeled parametrically (AFT), and an AFT model with the true φ plugged in (AFT-φ). Two sample sizes, n=50 and n=100, were considered.

Based on the simulation results, the estimators using the CV and GCV methods give similar results, so the results using the GCV method were reported, which was significantly faster than the CV method. Table 5 summarizes the mean bias of ν, the standard deviation (SD) of ν and means squared errors (MSE) over 200 Monte Carlo data sets. In all cases, the estimator of ν outperforms the other estimators in terms of MSE and its performance is comparable with the estimator using true. The number of knots has little impact on the performance of the proposed estimator. The usual AFT estimator of u exhibits the largest bias and MSE when is not linear, which indicates that it is important to adjust for the nonlinear effect of X even when one is only interested in the effect of Z. While the stratification step in the S_{K}-AFT method results into improved performance, it still under-performs the estimator, as the number of strata changes, its performance can vary considerably and it is not obvious how to choose the number of strata in practice.

TABLE 5
Simulation results for parameter estimation (υ) where d = 1 and υ = 1.
PL-AFT, the partly linear AFT model with r knots; S_{K}-AFT, the stratified
AFT estimator with K strata; AFT, the usual AFT model with both Xᵢ and
Zᵢ modeled parametrically; and AFT-φ, the AFT model with
true φ plugged in.

| φ(X) = 2X | | | φ(X) = 2X² | | |
|---|---|---|---|---|---|
| Bias | SD | MSE | Bias | SD | MSE |

n = 50

| | | | | | | |
|---|---|---|---|---|---|---|
| PL-AFT (r = 2) | −0.012 | 0.159 | 0.025 | −0.002 | 0.166 | 0.028 |
| PL-AFT (r = 4) | −0.010 | 0.159 | 0.025 | −0.001 | 0.168 | 0.028 |
| S₅-AFT | 0.095 | 0.288 | 0.092 | −0.065 | 0.436 | 0.195 |
| S₁₀-AFT | 0.028 | 0.223 | 0.050 | −0.043 | 0.299 | 0.091 |
| S₂₅-AFT | 0.031 | 0.303 | 0.093 | −0.038 | 0.381 | 0.146 |
| AFT | −0.004 | 0.153 | 0.023 | 0.021 | 1.214 | 1.475 |
| True-φ | −0.007 | 0.154 | 0.024 | −0.005 | 0.158 | 0.025 |

n = 100

| | | | | | | |
|---|---|---|---|---|---|---|
| PL-AFT (r = 2) | −0.009 | 0.113 | 0.013 | −0.002 | 0.115 | 0.013 |
| PL-AFT (r = 4) | −0.009 | 0.113 | 0.013 | −0.001 | 0.115 | 0.013 |
| S₅-AFT | 0.044 | 0.163 | 0.029 | −0.023 | 0.210 | 0.045 |
| S₁₀-AFT | 0.001 | 0.157 | 0.025 | −0.009 | 0.185 | 0.034 |
| S₂₅-AFT | −0.007 | 0.193 | 0.037 | 0.008 | 0.209 | 0.044 |
| AFT | −0.008 | 0.113 | 0.013 | 0.071 | 0.755 | 0.575 |
| AFT-φ | −0.009 | 0.113 | 0.013 | −0.002 | 0.111 | 0.012 |

### Feature Selection and Prediction

In the second set of simulation studies, the case where the regression function consists of nonlinear effect of a single covariate Xᵢ was still considered, but the dimension of the linear effects via Zᵢ were increased. The simultaneous estimation and feature selection problem was focused on for Zᵢ as well as the prediction performance when using both Zᵢ and Xᵢ. The true regression coefficients are set at ν=(1, 1, 0, 0, 0, 1, 0, 0)′, which corresponds to a strong signal or effect size, and ν=(0.5, 0.5, 0, 0, 0, 0.5, 0, 0)′, which corresponds to a weak signal or effect size. εᵢ follows N(0, σ²) with σ²=1 and is mutually independent of (Xᵢ; Zᵢ). The predictors followed a standard normal with the correlation between the jth and kth components of Zᵢ equal to ρ and ρ=0, 0.5, 0.9 were considered in the simulation. The random variable Xi was correlated to Zi through the relation Xᵢ=0.5Z₁ᵢ+0.5Z₂ᵢ+0.5Z₃ᵢ+Ui, where Uᵢ is Un (−1, 1) and completely independent of all other random variables. φ(Xᵢ)=(0.2*Xᵢ+0.5*Xᵢ²+0.15*Xᵢ³)I(Xᵢ≧0)+(0.05*Xᵢ)I(Xᵢ<0) was considered, where (•) is the indicator function. This setup mimics a practical setting where there is little change in the outcome variable when the clinical variable (X) is less than a threshold level (X=0), but as X increases past the threshold level, the outcome variable increases at a considerably higher rate. The censoring random variable was simulated according to the rule, Cᵢ=φ(Xᵢ)+Zᵢν+Uᵢ*, where follows the uniform distribution Un (0, τ) with τ=6. The resulting proportion of censoring ranges from 20% to 30%.

Five models were compared: (1) the Lasso partly linear AFT model (Lasso-PL) with r=6 which was fit using the loss function (7); (2) the Lasso stratified model (Lasso-S_{K}) where K denotes the number of strata, which was an extension to combine the stratified model in Chen et al. (Chen et al., Statistica Sinica 15:767-79 (2005)) with Lasso; (3) the Lasso linear AFT model assuming a linear effect for both X and Z (Lasso-L); (4) the usual AFT parametric model assuming a linear effect for both with regularization (AFT); and (5) the so-called oracle partly linear model (Oracle) with ν₃, ν₄, ν₅, ν₇, and ν₈ fixed at 0 and r=6 for the penalized splines. The oracle model while unavailable in practice, may serve as the optimal bench mark for the purpose of comparisons. In each instance of lasso, the GCV method was used to tune the regularization parameters, λ and γ.

In each simulation run, a training data set was generated of size n=125 to estimate the parameters of interest and a testing data set of size 10n to evaluate the prediction performance of the partly linear model. To evaluate the performance of parameter estimation, the sum squared error (SSE) of [symbol] defined as ( [symbol] −ν)^{T}( [symbol] −ν) was monitored. To evaluate the performance of model selection, the proportion of correct P_{C}≡Σ^{d}ᵢ₌₁ I( [symbol] =0)I(νᵢ=0)/Σ^{d}ᵢ₌₁ I(νᵢ=0) and incorrect (P_{I}≡Σ^{d}ᵢ₌₁ I( [symbol] ᵢ=0)I(νᵢ≠0)/Σ^{d}ᵢ₌₁ I(ν_{i≠}0)) zeroes was monitored. To assess the prediction performance of the partly linear model, two mean squared prediction errors, MSPE₁=Σ¹⁰ⁿⱼ₌₁[φ(Xⱼ)−φ(Xⱼ)+( [symbol] −ν)^{T}Zⱼ]², and MSPE₂=Σ¹⁰ⁿⱼ₌₁[( [symbol] −ν)^{T}Zⱼ]² were computed, where j goes through the observations in the testing data set. MSPE₁ is the squared prediction error using both nonparametric and parametric components in (1), and MSPE₂ is the squared prediction error using only parametric components in (1), both of which are of potential interest in practice. Note that the stratified Lasso model does not provide an estimate of the effect of X, therefore MSPE₁ is not applicable for Lasso-SK. For each simulation setting, these measures were averaged over 200 Monte Carlo data sets. The simulation results are summarized in Table 6. For the Lasso stratified model, K=2, 4, 8, 10, and 25 were considered. In all cases, K=4 provides the best results; hence Table 6 only presents the results for K=2, 4, and 8.

TABLE 6
Simulation results for feature selection and prediction where n = 125. Lasso-PL, the
Lasso partly linear AFT model with r = 6 knots; Lasso-S_{K}, the Lasso stratified
model with K strata; Lasso-L, the Lasso linear AFT model assuming a linear effect
for both Xᵢ and Zᵢ; AFT, the usual AFT parametric model assuming a linear effect for both
Xᵢ and Zᵢ without regularization; Oracle, the so-called oracle partly linear model with zero
coefficients being set to 0 and r = 6 for the penalized spline. P_{C}, the proportion of correct zero
estimates; P_{I}, the proportion of incorrect zero estimates.

| υ = (1, 1, 0, 0, 0, 1, 0, 0)′ | | | | | υ = (0.5, 0.5, 0, 0, 0, 0.5, 0, 0)′ | | | | |
|---|---|---|---|---|---|---|---|---|---|
| SSE | P_{C} | P_{I} | MSPE₁ | MSPE₂ | SSE | P_{C} | P_{I} | MSPE₁ | MSPE₂ |

ρ = 0

| | | | | | | | | | | |
|---|---|---|---|---|---|---|---|---|---|---|
| Lasso- | 0.009 | 0.708 | 0 | 0.277 | 0.070 | 0.008 | 0.744 | 0 | 0.267 | 0.065 |
| PL | 0.025 | 0.454 | 0 | NA | 0.202 | 0.022 | 0.472 | 0.002 | NA | 0.176 |
| Lasso-S₂ | 0.018 | 0.556 | 0 | NA | 0.146 | 0.014 | 0.588 | 0.002 | NA | 0.112 |
| Lasso-S₄ | 0.021 | 0.369 | 0 | NA | 0.165 | 0.020 | 0.464 | 0.007 | NA | 0.156 |
| Lasso-S₈ | 0.013 | 0.619 | 0 | 1.128 | 0.104 | 0.012 | 0.620 | 0.000 | 1.120 | 0.099 |
| Lasso-L | 0.018 | 0 | 0 | 1.135 | 0.148 | 0.018 | 0 | 0 | 1.126 | 0.139 |
| AFT | 0.004 | 1 | 0 | 0.361 | 0.029 | 0.004 | 1 | 0 | 0.222 | 0.030 |
| Oracle | | | | | | | | | | |

ρ = 0.5

| | | | | | | | | | | |
|---|---|---|---|---|---|---|---|---|---|---|
| Lasso- | 0.011 | 0.771 | 0 | 0.487 | 0.077 | 0.011 | 0.794 | 0.003 | 0.300 | 0.071 |
| PL | 0.039 | 0.404 | 0 | NA | 0.349 | 0.038 | 0.403 | 0.003 | NA | 0.341 |
| Lasso-S₂ | 0.021 | 0.575 | 0 | NA | 0.173 | 0.020 | 0.593 | 0.005 | NA | 0.145 |
| Lasso-S₄ | 0.026 | 0.561 | 0 | NA | 0.216 | 0.025 | 0.587 | 0.017 | NA | 0.200 |
| Lasso-S₈ | 0.019 | 0.718 | 0 | 3.055 | 0.129 | 0.018 | 0.746 | 0.013 | 3.167 | 0.118 |
| Lasso-L | 0.034 | 0 | 0 | 3.029 | 0.216 | 0.032 | 0 | 0 | 3.123 | 0.198 |
| AFT | 0.005 | 1 | 0 | 0.576 | 0.028 | 0.004 | 1 | 0 | 0.301 | 0.029 |
| Oracle | | | | | | | | | | |

ρ = 0.9

| | | | | | | | | | | |
|---|---|---|---|---|---|---|---|---|---|---|
| Lasso- | 0.046 | 0.744 | 0.003 | 0.402 | 0.110 | 0.039 | 0.760 | 0.128 | 0.417 | 0.135 |
| PL | 0.131 | 0.508 | 0.013 | NA | 0.624 | 0.108 | 0.495 | 0.175 | NA | 0.581 |
| Lasso-S₂ | 0.071 | 0.573 | 0.003 | NA | 0.158 | 0.060 | 0.591 | 0.134 | NA | 0.136 |
| Lasso-S₄ | 0.116 | 0.249 | 0.007 | NA | 0.310 | 0.096 | 0.444 | 0.128 | NA | 0.373 |
| Lasso-S₈ | 0.087 | 0.754 | 0.032 | 6.985 | 0.238 | 0.061 | 0.778 | 0.264 | 6.902 | 0.254 |
| Lasso-L | 0.208 | 0 | 0 | 6.869 | 0.321 | 0.221 | 0 | 0 | 6.784 | 0.355 |
| AFT | 0.017 | 1 | 0 | 0.501 | 0.050 | 0.017 | 1 | 0 | 0.584 | 0.053 |
| Oracle | | | | | | | | | | |

The simulations showed that the performance of the usual rank-based AFT estimator was not satisfactory in terms of both prediction and feature selection. In all cases, the Lasso partly linear AFT estimator exhibited substantially smaller SSE, MSPE₁, and MSPE₂ compared to other Lasso estimators and its MSPE₁ is comparable to that of the Oracle estimator, whereas the Lasso stratified estimators exhibited the worst performance. In terms of the feature selection, both the method described herein and the Lasso linear AFT method correctly identified the majority of the zero regression coefficients (P_{C}); the method described herein outperforms the Lasso linear AFT method when ρ=0 or 0.5 and their performances are comparable when the correlation becomes extremely high (ρ=0.9). Note that as ρ increases, so does the correlation between X and Z. By comparison, the Lasso stratified estimators only identify less than 30% of true zeros in some cases and roughly half of the true zeros in the rest of the cases. When there is no correlation and the signal is strong, all Lasso estimators successfully avoided setting nonzero coefficients to zero (P₁=0). However, as the correlation gets stronger and the signal becomes weaker, P₁ increases for all estimators; in particular, P₁ becomes appreciable for the Lasso linear AFT estimator when ρ=0.9, whereas it remains moderate for our estimator as well as some Lasso stratified estimator.

To summarize, the lasso partly linear AFT model achieved better performance in all three areas, estimation, feature selection, and predication. While the lasso stratified estimator performed reasonably well in estimation, its performance in feature selection and prediction was not satisfactory. When the effect of X is nonlinear, the performance of the Lasso linear AFT model deteriorates, and the deterioration can be substantial when prediction is of interest.

### Example 4

### Prostate Cancer Study Using Novel Partly Linear Accelerated Failure Time (AFT) Model

The methods described above were used to analyze data from a prostate cancer study. Data from 83 patients were used in this data analysis. The outcome of interest is time to prostate cancer recurrence, which begins on the surgery date of the prostatectomy and is subject to censoring; the observed survival time ranges from 2 months to 160 months and the censoring rate is 62.6%. In the data analysis, the log-transformed survival time was used to fit AFT models. Gene expression data were measured using 1536 probes from samples collected at the baseline, i.e., right after the surgery. In addition, two clinical variable, the PSA (Prostate Specific Antigen) and total gleason score, are of particular interest in this study and were measured for all subjects at baseline. The total gleason score only takes integer values from 5 to 9 and 89% of patients have a total gleason score of either 6 or 7; combining this with suggestions from the investigators, the total gleason score is dichotomized as >6 or not.

Before the data analysis, all gene expression measurements were preprocessed by investigators and then standardized by us to have mean 0 and unit standard deviation. In the literature, it is of interest to study both the gene data and probe data (Nakagawa et al., (2008)) and it can be argued that in some cases it is more important to examine the probe data. Cox PH models were first fit for each individual probe and ranked according to their score test statistics from the largest (J=1) to the smallest (J=1536). Subsequently, feature selection was conducted while adjusting for the nonlinear effect of PSA. To fit the models of interest, the top d=25 probes were selected to fit the lasso partly linear AFT model.

### Feature Selection

Four models were used to conduct feature selection, the Lasso-PL with r=8, Lasso-S_{K}, Lasso-L, and usual linear AFT without regularization. In the Lasso-PL model, Xᵢ in model (3) is PSA, which is modeled using penalized splines, and Z include both 1536 probes and the binary clinical variable gleason score. Similarly, in the Lasso stratified model, the stratification is based on PSA.

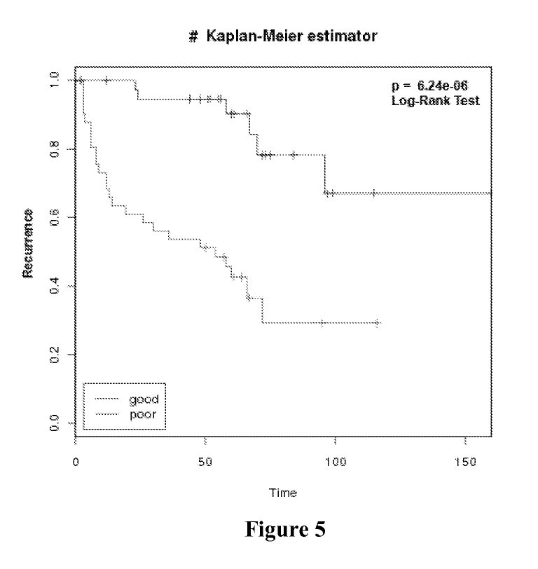

The results are summarized in Table 7. A linear effect of PSA was included in the Lasso linear AFT model and was estimated to be nonzero (−0.132), which further justifies the inclusion of PSA in the final model. On the other hand, the clinical variable, total gleason score, is not selected by any of the methods. FIG. 5 shows the estimated effect of PSA using the Lasso partly linear AFT module and it is evident that its effect is nonlinear. Specifically, the primary endpoint initially decreases as the PSA value increases and then starts to increase slightly at about PSA=11. After further examination of the data, the mean of PSA was found to be 8.20 (SD=4.13) with a range of 0-32; most patients had PSA values ranging from 0-15.2, but five had PSA values between 18-32, all of which had censored outcomes. As a result, the tail was suspected to be an artifact of the data. Additional analysis was conducted and the 5 outliers were removed. While the results for feature selection remain the same, the estimate φ of became more flat towards the right tail of the curve, which indicates that the effect of PSA levels off after PSA becomes greater than 11.

TABLE 7
Feature selection for the prostate cancer study, n = 83.

| | | | | | | |
|---|---|---|---|---|---|---|
| | Lasso- | Lasso- | | | | Linear |
| Predictor | PL | S₂ | Lasso-S₄ | Lasso-S₈ | Lasso-L | AFT |

Clinical Variable

| | | | | | | |
|---|---|---|---|---|---|---|
| PSA | φ | NA | NA | NA | −0.076 | −0.806 |
| Gleason | 0 | 0 | 0 | 0 | 0 | −0.390 |

Probe Variable

| | | | | | | |
|---|---|---|---|---|---|---|
| 1 | −0.202 | −0.272 | −0.166 | 0 | −0.251 | −0.387 |
| 2 | −0.128 | 0 | 0 | 0 | −0.163 | −0.891 |
| 3 | 0 | 0 | 0 | 0 | 0 | −0.245 |
| 4 | 0 | 0 | 0 | −0.107 | 0 | 0.063 |
| 5 | 0 | 0 | 0 | 0 | 0 | 1.079 |
| 6 | 0 | 0 | 0 | 0 | 0 | −0.116 |
| 7 | 0.310 | 0.363 | 0.210 | 0 | 0.300 | 0.342 |
| 8 | 0 | 0 | 0 | 0 | 0 | 0.238 |
| 9 | 0 | −0.139 | 0 | 0 | −0.040 | −0.620 |
| 10 | −0.048 | −0.025 | −0.102 | −0.058 | 0 | −0.307 |
| 11 | 0 | 0 | 0 | −0.055 | 0 | −0.197 |
| 12 | 0 | 0 | 0 | 0 | 0 | 0.039 |
| 13 | 0 | 0.019 | 0 | 0.063 | 0.135 | −0.174 |
| 14 | 0 | 0 | 0 | 0 | 0 | 0.131 |
| 15 | 0.539 | 0.567 | 0.584 | 0.372 | 0.655 | 0.890 |
| 16 | 0 | 0 | 0 | 0 | 0 | 0.058 |
| 17 | −0.143 | −0.060 | −0.180 | −0.100 | −0.140 | −0.713 |
| 18 | 0 | 0 | 0 | 0 | 0 | 0.436 |
| 19 | 0 | 0 | 0 | 0 | 0 | −0.060 |
| 20 | 0 | 0 | 0 | 0 | 0 | −0.485 |
| 21 | 0 | 0 | 0 | 0 | 0 | 0.250 |
| 22 | 0 | 0 | 0 | 0 | 0 | 0.842 |
| 23 | 0 | 0 | 0 | 0 | 0 | 0.141 |
| 24 | 0 | 0 | 0 | 0 | 0 | 0.257 |
| 25 | 0 | 0 | 0 | 0 | 0 | 0.557 |

As to feature selection, the majority of the top 25 probes were not selected by the methods considered herein. The results were similar using different methods, but they do select somewhat different sets of probes. Of the stratified Lasso estimators, it seemed that Lasso-S₄ provided the most consistent results with the Lasso-PL method, which seems to agree with the finding in the simulations that Lasso-S₄ achieved better performance compared to other K values. Among the probes picked by the Lasso-PL method, probe 1, 7, 15, and 17 were also selected by Lasso-S₄ and Lasso-L, and 2 was not selected by the Lasso-S₄ method and 10 was not selected by the Lasso-L method. In addition, the Lasso-L method also selected probes 9 and 13. This observation agreed with the simulation results, i.e., when the correlation was moderate, the Lasso-L method tended to select a larger number of incorrect features; and the difference between the Lasso-PL method and the Lasso-L method was attributed to the nonlinear effects of PSA.

A sensitivity analysis was conducted to examine the impact of d, where the feature selection procedures were repeated using the different numbers of top probes (d=20, 30, and 35). The results of the feature selection are summarized in Table 8, which uses the set of probes selected for d=25 as the reference set. When d=20, 25, and 30, the Lasso-PL model selected exactly the same subset of probes; and when d=35, the Lasso-PL model dropped probe 2 and selected probe 32. For all d's, the estimated nonlinear effect of PSA using the Lasso-PL model was almost identical. While the Lasso-S₄ and Lasso-S₈ models selected different sets of features compared to the Lasso-PL model, they were also insensitive to the value of d. By comparison, the Lasso-L model and Lasso-S₂ models seemed to be considerably more sensitive to the value of d. In particular, when d=20, the Lasso-L method selected a significantly larger number of probes, which seemed to indicate that the impact of the misspecified linear effect of PSA was substantial in terms of the feature selection, especially when a small number of probes were used.

TABLE 8
Sensitivity analysis for feature selection in the prostate cancer study
using the top d probes.

| d | Lasso-PL | Lasso-S₂ | Lasso-S₄ | Lasso-S₈ | Lasso-L |
|---|---|---|---|---|---|
| 25 | Reference | +: 9, 13 | +: | +: 4, 11, 13 | +: 9, 13 |
| | Set | −: 2 | −: 2 | −: 1, 2, 7 | −: 10 |
| 20 | +: | +: 8, 9, 13, 18, 20 | +: | +: 4, 11, 13 | +: 4, 5, 8, 9, 13, 16, 18, 20 |
| | −: | −: | −: 2 | −: 1, 2, 7 | −: 10 |
| 30 | +: | +: 9, 18, 27, 28 | +: | +: 4, 11, 13 | +: 9, 13 |
| | −: | −: | −: 2 | −: 1, 2, 7 | −: 10 |
| 35 | +: 32 | +: 9, 13, 32, 35 | +: 35 | +: 4, 11, 13 | +: 9, 13, 32, 35 |
| | −: 2 | −: 2, 10, 17 | −: 2 | −: 1, 2, 7 | −: 10 |

The set of probes selected by Lasso-PL with d = 25 is considered the reference set, {1, 2, 7, 10, 15, 17}.
+, probes not in the reference set that are selected by a method;
−, probes in the reference set that are not selected by a method.

### Prediction Performance

To internally evaluate the prediction performance of the models of interest, an approach was followed as used in Cai et al. (Cai et al., Biometrics, In Press (2009)). The data were randomly split into a training sample (70%) and a validation sample (30%). The models were fit using the training sample and were then used to predict the risk of failure for subjects in the validation sample. The subjects were classified as high or low risk based on whether the predicted risk exceeded the median risk. Subsequently, a log-rank test was conducted in the validation sample comparing the two risk groups. This procedure was repeated 1000 times and the prediction performance was evaluated using the resulting p-values. The models were compared that were used in the data analysis, namely, the Lasso-PL with r=8, Lasso-L, and usual linear AFT, all of which use both clinical variables and gene expression data. Note that the Lasso-S_{K} can not be used for prediction. Two other models were also considered that use only the clinical variables, namely, a partly linear AFT model that models PSA nonparametrically and a linear AFT model.

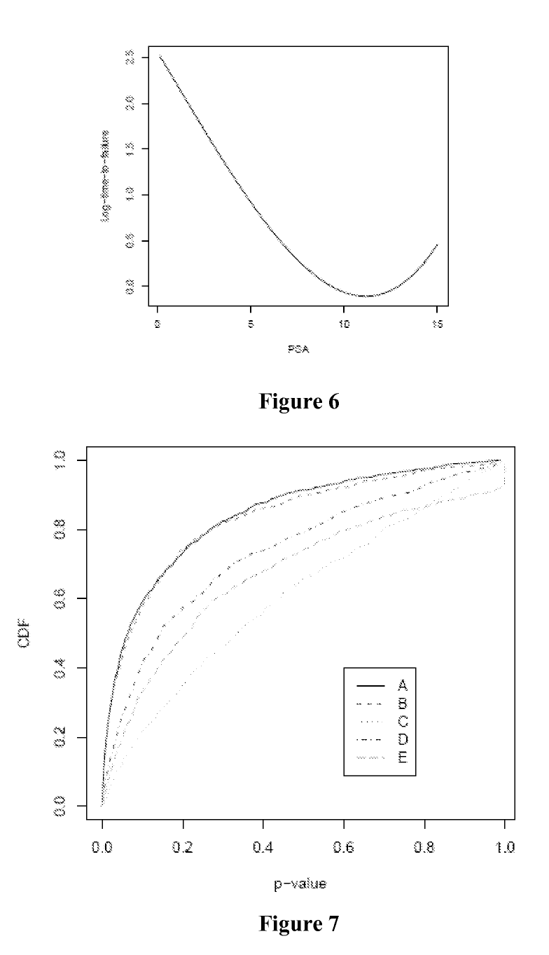

The results were visualized using the cumulative distribution function of the p-values from the log-rank tests in FIG. 6. In FIG. 6, the larger the area under the curve is, the better the performance of the method. In addition, the proportion of p-values being less than 0.05 are computed for predictions based on three models that use all data, namely, the Lasso-PL with r=8 (45:3%), the Lasso-L (43:0%), and the usual linear AFT (12:6%), as well as two models that use only clinical variables, namely, a partly linear AFT model (26:3%) and a linear AFT model (19:2%). Our results show that it is important to correctly specify the nonlinear effect of PSA when prediction is of interest. In the absence of gene expression data, the partly linear AFT model (D in FIG. 6) achieved considerably better performance than the linear AFT model (E in FIG. 6). After adjusting for gene expression data, while the prediction performance of the lasso partly linear AFT model (A in FIG. 6) was still slightly better than that of the lasso linear AFT model (B in FIG. 6), the improvement diminished. A possible explanation was that the gene expression data were potentially correlated with the PSA level and consequently the addition of gene expression data was able to offset the impact of the misspecified linear effect of PSA, especially when the prediction performance was evaluated based on the dichotomized risk scores.

The results provide the answer to the research question of interest, whether the addition of gene expression data improved the prediction performance of the resultant risk scores. If the appropriate models were used (e.g., the Lasso partly linear AFT model), the prediction performance improved substantially when the gene expression data were added (A in FIG. 6) compared to the AFT models without using the gene expression data (D and E in FIG. 6). However, if an inappropriate model was used, the gain in prediction performance was not realized. Specifically, the linear AFT model without regularization that used both clinical and gene expression data (C in FIG. 6) underperformed the AFT models that used only clinical variables (D and E in FIG. 6).

1. A method of predicting the recurrence, progression, and metastatic potential of a prostate cancer in a subject, the method comprising detecting in a sample from the subject three or more biomarkers selected from the group consisting of FOXO1A, SOX9, CLNS1A, PTGDS, XPO1, LETMD1, RAD23B, ABCC3, APC, CHEST, EDNRA, FRZB, HSPG2, and TMPRSS2_ETV1 FUSION, an increase or decrease in one or more of these biomarkers as compared to a standard indicating a recurrent, progressive, or metastatic prostate cancer.

2. The method of claim 1, wherein the sample comprises prostate tumor tissue.

3. The method of claim 1, wherein the detecting step comprises detecting mRNA levels of the biomarker.

4. The method of claim 3, wherein the RNA detection comprises reverse-transcription polymerase chain reaction (RT-PCR) assay; quantitative real-time-PCR (qRT-PCR); Northern analysis; microarray analysis; and cDNA-mediated annealing, selection, extension, and ligation (DASL®) assay.

5. (canceled)

6. The method of claim 1, wherein multiple biomarkers are detected and wherein the detection comprises identifying an RNA expression pattern.

7-18. (canceled)

19. The method of claim 1, further comprising detecting one or more biomarkers selected from the group consisting of miR-103, miR-339, miR-183, miR-182, miR-136, and miR-221.

20-23. (canceled)

24. A method of predicting the recurrence, progression, and metastatic potential of a prostate cancer in a subject, the method comprising detecting in a sample from a subject one or more biomarkers selected from the group consisting of miR-103, miR-339, miR-183, miR-182, miR-136, and miR-221, an increase or decrease in one or more of these biomarkers as compared to a standard indicating a recurrent, progressive, or metastatic prostate cancer.

25-28. (canceled)

29. A method of treating a subject with prostate cancer comprising modifying a treatment regimen of the subject based on the results of the method of claim 1.

30. The method of claim 29, wherein the treatment regimen is modified to be aggressive based on an increase in one or more biomarkers selected from the group consisting of CLNS1A, XPO1, LETMD1, RAD23B, TMPRSS2_ETV1 FUSION, ABCC3, SPC, CHES1, FRZB, HSPG2, miR-103, miR-339, miR-183, and miR-182 as compared to a standard, and a decrease in one or more biomarkers selected from the group consisting of FOXO1A, SOX9, PTGDS, EDNRA, miR-136, and miR-221 as compared to a standard.

31-41. (canceled)

43. An array consisting of probes to three or more of the biomarkers selected from the group consisting of FOXO1A, SOX9, CLNS1A, PTGDS, XPO1, LETMD1, RAD23B, ABCC3, APC, CHES1, EDNRA, FRZB, HSPG2, TMPRS S2 ETV1 FUSION, miR-103, miR-339, miR-183, miR-182, miR-136, and miR-221.

44. The method of claim 1, wherein the biomarkers are FOXO1A, SOX9, CLNS1A, PTGDS, XPO1, LETMD1, RAD23B, and TMPRSS2_ETV1 FUSION.
